(19) United States
(12) Patent Application Publication
(10) Pub. No.: US 2014/0045860 A1
(43) Pub. Date: Feb. 13, 2014

(54) SMALL MOLECULE INHIBITORS FOR TREATING PARASITIC INFECTIONS

(71) Applicants: NEW YORK UNIVERSITY, New York, NY (US); THE BROAD INSTITUTE, INC., Cambridge, MA (US)

(72) Inventors: Leigh C. Carmody, Allston, MA (US); Ana Rodriguez, Westbury, NY (US); Esther Bettiol, Geneva (CH); Michelle Palmer, Harvard, MA (US); Andrew Germain, Somerville, MA (US); Benito Munoz, Newtonville, MA (US); Sivaraman Dandapani, Malden, MA (US); Michael Foley, Chestnut Hill, MA (US)

(73) Assignees: NEW YORK UNIVERSITY, New York, NY (US); THE BROAD INSTITUTE, INC., Cambridge, MA (US)

(21) Appl. No.: 13/965,685

(22) Filed: Aug. 13, 2013

Related U.S. Application Data
(63) Continuation of application No. PCT/US2012/025071, filed on Feb. 14, 2012.
(60) Provisional application No. 61/442,509, filed on Feb. 14, 2011.

(51) Int. Cl. C07D 413/12; C07D 498/04; C07D 413/14

(52) U.S. Cl. CPC C07D 413/12; C07D 413/14; C07D 498/04
USPC 514/255.05; 540/455; 514/338; 514/302

(57) ABSTRACT

The invention relates to a compound of Formula I or a pharmaceutically acceptable ester, salt, prodrug or metabolite thereof;

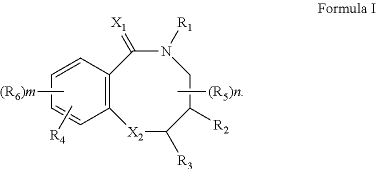

### RELATED APPLICATIONS

This application is a continuation of International Application No. PCT/US2012/025071, which designated the United States and was filed on Feb. 14, 2012, published in English, which claims the benefit of U.S. Provisional Application No. 61/442,509, filed on Feb. 14, 2011. The entire teachings of the above applications are incorporated herein by reference.

### GOVERNMENT SUPPORT

The invention was made with government support under U54-HG005032, awarded by the National Institutes of Health and under 1R03-MH085673-01, awarded by the National Institutes of Health. The Government has certain rights in the invention.

### FIELD OF THE INVENTION

This invention relates to small molecule pharmaceutical compounds and to their uses for treatment of parasitic infections, in particular as antitrypansomal therapeutic compounds to treat infections with protozoa, most particularly Trypanosoma cruzi.

### BACKGROUND OF THE INVENTION

Trypanosomatids are a group of kinetoplastid protozoa distinguished by having only a single flagellum. Trypanosomatids are responsible for human diseases such as South American trypanosomiasis (Chagas Disease) caused by Trypanosoma cruzi and African trypanosomiasis (Sleeping Sickness) caused by Trypansoma brucei. These diseases are predominately diseases of the third world.

Chagas disease is endemic to 18 Latin American countries, with 13 million people chronically infected. Approximately 30% of chronically infected patients will suffer from irreversible damage to the heart and digestive tract leading to death.

Sleeping Sickness is endemic in some regions of sub-Saharan Africa, covering approximately 36 countries. It is estimated that 50,000-70,000 people are currently infected however it is also believed that many cases go unreported. The disease has 2 stages and without treatment is fatal. The first stage is characterized by fever, headaches, joint pain and itching. In the second phase of the disease the parasite invades the central nervous system by passing through the blood brain barrier and causes progressive mental deterioration leading to coma and death. Resistance is a problem with current treatments. In addition, current treatments are dosed via IV, which is difficult in many areas and adverse reaction to treatment can be severe and even life threatening.

In addition, Malaria is present in over 100 countries with more than 300 million new cases reported a year. 50% of malaria cases are caused by Plasmodium Falciparum. Plasmodium falciparum resistance has developed to many currently available malaria treatments.

### SUMMARY OF THE INVENTION

The invention relates to a compound of Formula I or a pharmaceutically acceptable ester, salt, prodrug or metabolite thereof;

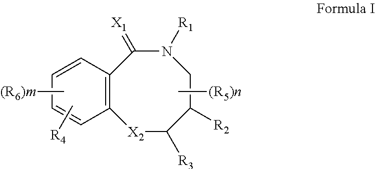

wherein
n is 0, 1, 2, 3,4 or 5;
m is 0, 1, 2, 3 or 4;
X₁ is O or S;
X₂ is O, S, S(O) or S(O)₂;
R₁ is hydrogen, alkyl, substituted alkyl, alkenyl, substituted alkenyl, alkynyl, substituted alkynyl, alkoxy, substituted alkoxy, alkylhydroxy, substituted alkylhydroxy, alkylamino, substituted alkylamino, alkylthio or substituted alkylthio; each R₂, R₃, R₅ and R₆ is independently selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀, —SR₁₀, —NR₁₀R₁₁, —C(O)R₁₀, —C(O)OR₁₀, —C(O)NR₁₀R₁₁, —N(R₁₀)C(O)R₁₁, —CF₃, —CN, —NO₂, —N₃, acyl, alkoxy, substituted alkoxy, alkylamino, substituted alkylamino, dialkylamino, substituted dialkylamino, alkylthio or substituted alkylthio;
   wherein each R₁₀ and R₁₁ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₀ and R₁₁ together with the atoms to which they are attached and any intervening atoms may form an additional optionally substituted, 3, 4, 5, 6 or 7 membered ring; and,
R₄ is selected from hydrogen, halogen, alkyl, substituted alkyl, alkenyl, substituted alkenyl, alkynyl, substituted alkynyl, aliphatic, substituted aliphatic, aryl, substituted aryl, —OR₁₀, —SR₁₀, —NR₁₀R₁₁, —C(O)R₁₀, —C(O)OR₁₀, —C(O)NR₁₀R₁₁, —N(R₁₀)C(O)R₁₁, —CF₃, —CN, —NO₂, —N₃.

The invention further relates to the treatment of a parasitic infection comprising the step of administering a compound of Formula I to a subject in need thereof. In a preferred embodiment, the invention relates to the treatment of a disease or disorder caused by Trypanosomatids comprising the step of administering a compound of Formula I to a subject in need thereof. In a more preferred embodiment, the disease or disorder is caused by Trypanosoma cruzi or Trypanosoma brucei. In a preferred embodiment, the disease or disorder is selected from Chagas disease, sometimes referred to as South American trypanosomiasis or African trypanosomiasis. In a more preferred embodiment, the disease is Chagas disease.

### DETAILED DESCRIPTION OF THE INVENTION

The invention relates to a compound of Formula I or a pharmaceutically acceptable ester, salt, prodrug or metabolite thereof;

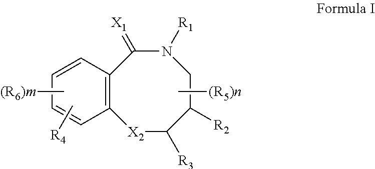

wherein
n is 0, 1, 2, 3, 4 or 5;
m is 0, 1, 2, 3 or 4;
X₁ is O or S;
X₂ is O, S, S(O) or S(O)₂;
R₁ is hydrogen, alkyl, substituted alkyl, alkenyl, substituted alkenyl, alkynyl, substituted alkynyl, alkoxy, substituted alkoxy, alkylhydroxy, substituted alkylhydroxy, alkylamino, substituted alkylamino, alkylthio or substituted alkylthio; each R₂, R₃, R₅ and R₆ is independently selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀, —SR₁₀, —NR₁₀R₁₁, —C(O)R₁₀, —C(O)OR₁₀, —C(O)NR₁₀R₁₁, —N(R₁₀)C(O)R₁₁, —CF₃, —CN, —NO₂, —N₃, acyl, alkoxy, substituted alkoxy, alkylamino, substituted alkylamino, dialkylamino, substituted dialkylamino, alkylthio or substituted alkylthio;
   wherein each R₁₀ and R₁₁ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₀ and R₁₁ together with the atoms to which they are attached and any intervening atoms may form an additional optionally substituted, 3, 4, 5, 6 or 7 membered ring; and,
R₄ is selected from hydrogen, halogen, alkyl, substituted alkyl, alkenyl, substituted alkenyl, alkynyl, substituted alkynyl, aliphatic, substituted aliphatic, aryl, substituted aryl, —OR₁₀, —SR₁₀, —NR₁₀R₁₁, —C(O)R₁₀, —C(O)OR₁₀, —C(O)NR₁₀R₁₁, —N(R₁₀)C(O)R₁₁, —CF₃, —CN, —NO₂, —N₃.

The invention further relates to the treatment of a parasitic infection comprising the step of administering a compound of Formula I to a subject in need thereof. In a preferred embodiment, the invention relates to the treatment of a disease or disorder caused by Trypanosomatids comprising the step of administering a compound of Formula I to a subject in need thereof. In a more preferred embodiment, the disease or disorder is caused by Trypanosoma cruzi or Trypanosoma brucei. In a preferred embodiment, the disease or disorder is Chagas disease.

In a preferred embodiment, X₁ is O. In a preferred embodiment, X₂ is O.

In a preferred embodiment, R₁ is selected from C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₁-C₁₂-alkylhydroxy, substituted C₁-C₁₂-alkylhydroxy, C₂-C₁₂-alkenylhydroxy, substituted C₂-C₁₂-alkenylhydroxy, C₂-C₁₂-alkynylhydroxy, substituted C₂-C₁₂-alkynylhydroxy, C₁-C₁₂-alkylthio, substituted C₁-C₁₂-alkylthio, C₂-C₁₂-alkenylthio, substituted C₂-C₁₂-alkenylthio, C₂-C₁₂-alkynylthio, substituted C₂-C₁₂-alkynylthio, C₃-C₁₂ cycloalkyl and substituted C₃-C₁₂-cycloalkyl.

In a preferred embodiment, the invention relates to a compound of Formula I wherein R₂ is selected from hydrogen, halogen, C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₅-C₁₂ aryl, substituted C₅-C₁₂ aryl C₃-C₁₂ cycloalkyl and substituted C₃-C₁₂-cycloalkyl.

In a preferred embodiment, R₃ is -G₁-X₃;
wherein G₁ is absent, C₁-C₁₂-alkyl, substituted C₁-C₁₂-alkyl, C₂-C₁₂-alkenyl, substituted C₂-C₁₂-alkenyl, C₂-C₁₂-alkynyl or substituted C₂-C₁₂-alkynyl; and, X₃ is —NR₁₂R₁₃, —OR₁₂, —SR₁₂, —C(O)R₁₂, —C(O)OR₁₂, —C(O)NR₁₂R₁₃, —N(R₁₂)C(O)R₁₃, —S(O)R₁₂ or S(O)₂R₁₂;
   wherein each R₁₂ and R₁₃ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₂ and R₁₃ together with the atoms to which they are attached and any intervening atoms may form an additional optionally substituted, 3, 4, 5, 6 or 7 membered ring.

In a preferred embodiment, R₁₂ is C₁-C₁₂-alkylaryl, substituted C₁-C₁₂-alkylaryl, C₂-C₁₂-alkenylaryl, substituted C₂-C₁₂-alkenylaryl, C₂-C₁₂-alkynylaryl or substituted C₂-C₁₂-alkynylaryl.

In a preferred embodiment, R₁₃ is hydrogen, methyl, ethyl, propyl or isopropyl.

In a preferred embodiment, R₁₂ is selected from Table A:

TABLE A

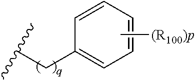

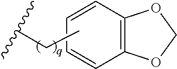

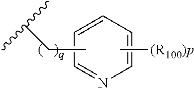

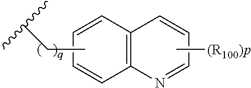

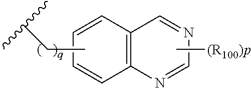

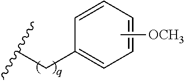

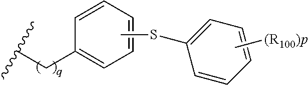

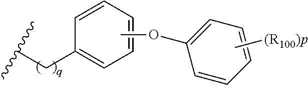

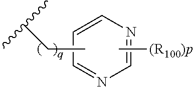

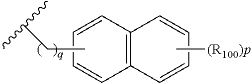

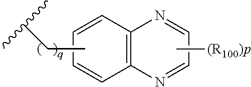

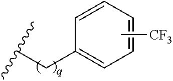

wherein q is 0, 1, 2, 3, 4, 5, or 6;
p is 0, 1, 2, 3, 4 or 5;
R₁₀₀ is selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀₁, —SR₁₀₁, —NR₁₀₁R₁₀₂, —C(O)R₁₀₁, —C(O)OR₁₀₁, —C(O)NR₁₀₁R₁₀₂, —N(R₁₀₁)C(O)R₁₀₂, —CF₃, —CN, —NO₂, —N₃, acyl, alkoxy, substituted alkoxy, alkylamino, substituted alkylamino, dialkylamino, substituted dialkylamino, alkylthio or substituted alkylthio;
   wherein each R₁₀₁ and R₁₀₂ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₀₁ and R₁₀₂ together with the atoms to which they are attached and any intervening atoms may form an additional optionally substituted, 3, 4, 5, 6 or 7 membered ring.

In a preferred embodiment, R₄ is —X₄-G₂;
wherein X₄ is absent, C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, —O—, —S—, —C(O)—, —C(O)O—, —C(O)NR₁₄—, —N(R₁₄)C(O)—, —S(O)— or S(O)₂—;
   wherein R₁₄ is selected from hydrogen, alkyl, substituted alkyl, alkenyl, substituted alkenyl, alkynyl, substituted alkynyl, aliphatic, substituted aliphatic, aryl and substituted aryl; and,
G₂ is aryl or substituted aryl.

In a more preferred embodiment, R₄ is —X₄-G₂ wherein —X₄— is —NHC(O)—, —C(O)N(CH₃)—-C(O)—, —C(O)O— or —NH—;

In a preferred embodiment, G₂ is selected from Table B:

TABLE B

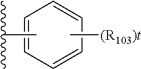

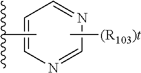

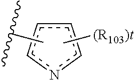

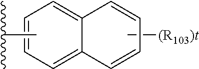

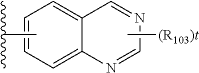

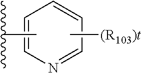

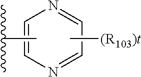

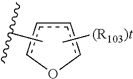

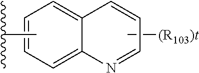

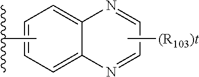

t is 0, 1, 2, 3, 4 or 5;
R₁₀₃ is selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀₁, —SR₁₀₄, —NR₁₀₄R₁₀₅, —C(O)R₁₀₄, —C(O)OR₁₀₄, —C(O)NR₁₀₄R₁₀₅, —N(R₁₀₄)C(O)R₁₀₅, —CF₃, —CN, —NO₂, —N₃, acyl, alkoxy, substituted alkoxy, alkylamino, substituted alkylamino, dialkylamino, substituted dialkylamino, alkylthio or substituted alkylthio;
   wherein each R₁₀₄ and R₁₀₅ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two R₁₀₄ and R₁₀₅ groups together with the atoms to which they are attached and any intervening atoms may form an additional optionally substituted, 3, 4, 5, 6 or 7 membered ring.

In a more preferred embodiment, R₁ is C₁-C₈-alkyl, C₁-C₈-alkenyl or C₁-C₈-alkynyl. In a more preferred embodiment, R₁ is a C₁-C₄-alkyl C₁-C₄-alkenyl or C₁-C₄-alkynyl. In a more preferred embodiment, R₁ is a C₁-C₄-alkylhydroxy, substituted C₁-C₄-alkylhydroxy, C₂-C₄-alkenylhydroxy, substituted C₂-C₄-alkenylhydroxy, C₂-C₄-alkynylhydroxy, substituted C₂-C₄-alkynylhydroxy, C₁-C₄-alkylthio, substituted C₁-C₄-alkylthio, C₂-C₄-alkenylthio, substituted C₂-C₄-alkenylthio, C₂-C₄-alkynylthio, substituted C₂-C₄-alkynylthio.

In a more preferred embodiment, R₁ is selected from Table C:

TABLE C

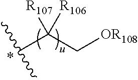

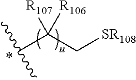

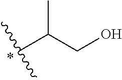 [SMILES: *C(C)CO]

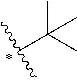 [SMILES: *C(C)(C)C]

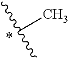

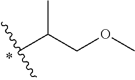 [SMILES: *C(C)COC]

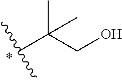 [SMILES: *C(C)(C)CO]

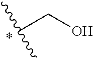 [SMILES: *CO]

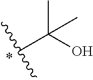 [SMILES: *C(C)(C)O]

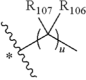

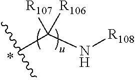

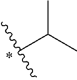 [SMILES: *C(C)C]

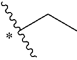 [SMILES: *CC]

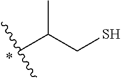 [SMILES: *C(C)CS]

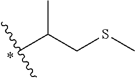 [SMILES: *C(C)CSC]

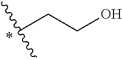 [SMILES: *CCO]

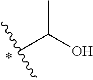 [SMILES: *C(C)O]

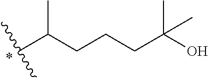 [SMILES: *C(C)CCCC(C)(C)O]

u is 0, 1, 2, 3, 4, 5, 6, 7, 8, 9 or 10;
each R₁₀₆, R₁₀₇ and R₁₀₈ is independently selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀₉, —SR₁₀₉, —NR₁₀₉R₁₁₀, —C(O)R₁₀₉, —C(O)OR₁₀₉, —C(O)NR₁₀₉R₁₁₀, —N(R₁₀₉)C(O)R₁₁₀, —CF₃, —CN, —NO₂, —N₃, acyl, alkoxy, substituted alkoxy, alkylamino, substituted alkylamino, dialkylamino, substituted dialkylamino, alkylthio or substituted alkylthio;

wherein each R₁₀₉ and R₁₁₀ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two R₁₀₉ and R₁₁₀ groups together with the atoms to which they are attached and any intervening atoms may form an additional optionally substituted, 3, 4, 5, 6 or 7 In a preferred embodiment, the invention relates to a compound of Formula IA or a pharmaceutically acceptable ester, salt, prodrug or metabolite thereof;

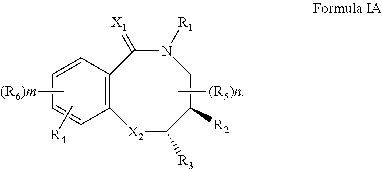

In a preferred embodiment, the invention relates to a compound of Formula II or a pharmaceutically acceptable ester, salt, prodrug or metabolite thereof;

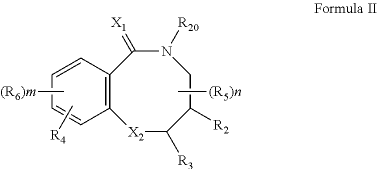

wherein R₂₀ is hydrogen, alkyl, alkenyl or alkynyl, preferably C₁-C₄ alkyl.

In a preferred embodiment, the invention relates to a compound of Formula III or a pharmaceutically acceptable ester, salt, prodrug or metabolite thereof;

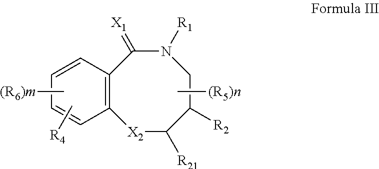

wherein R₂₁ is -G₂₀-X₂₀;
wherein G₂₀ is absent, C₁-C₁₂-alkyl, substituted C₁-C₁₂-alkyl, C₂-C₁₂-alkenyl, substituted C₂-C₁₂-alkenyl, C₂-C₁₂-alkynyl or substituted C₂-C₁₂-alkynyl; X₂₀ is —NR₃₀R₃₁, —OR₃₀, —SR₃₀, —C(O)R₃₀, —C(O)OR₃₀, —C(O)NR₃OR₃₁, —N(R₃₀)C(O)R₃₁, —S(O)R₃₀ or S(O)₂R₃₁;
   wherein R₃₀ is selected from absent, hydrogen, alkyl, substituted alkyl, alkenyl, substituted alkenyl, alkynyl, substituted alkynyl, aliphatic, substituted aliphatic, aryl and substituted aryl; and,
   R₃₁ is aryl substituted with 1, 2, 3, 4 or 5 halogens, or heteroaryl substituted with 1, 2, 3, 4 or 5 halogens.

In one embodiment, the invention relates to a compound of Formula IV or a pharmaceutically acceptable ester, salt, prodrug or metabolite thereof;

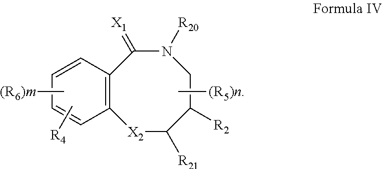

In one embodiment, the invention relates to a compound of Formula V or a pharmaceutically acceptable ester, salt, prodrug or metabolite thereof;

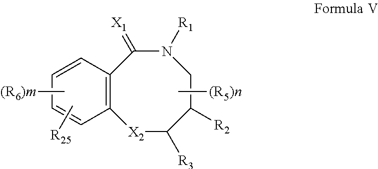

wherein R₂₅ is aryl, substituted aryl, heteroaryl, substituted heteroaryl, heterocyclic or substituted heterocyclic.

In a preferred embodiment, R₂₅ is aryl, substituted aryl, heterocyclic or substituted heterocylic and m is zero.

In a preferred embodiment, R₂₅ is selected from:

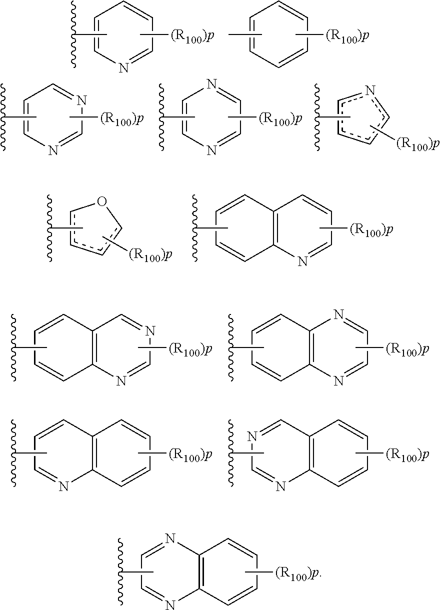

The invention further relates to the treatment of a parasitic infection comprising the step of administering a compound of Formula I-IV to a subject in need thereof. In a preferred embodiment, the invention relates to the treatment of a disease or disorder caused by Trypanosomatids comprising the step of administering a compound of Formula I-IV to a subject in need thereof. In a more preferred embodiment, the disease or disorder is caused by Trypanosoma cruzi or Trypanosoma brucei. In a preferred embodiment, the disease or disorder is Chagas disease.

In a preferred embodiment, X₁ is O. In a preferred embodiment, X₂ is O.

In a preferred embodiment, R₁ is selected from C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₁-C₁₂-alkylhydroxy, substituted C₁-C₁₂-alkylhydroxy, C₂-C₁₂-alkenylhydroxy, substituted C₂-C₁₂-alkenylhydroxy, C₂-C₁₂-alkynylhydroxy, substituted C₂-C₁₂-alkynylhydroxy, C₁-C₁₂-alkylthio, substituted C₁-C₁₂-alkylthio, C₂-C₁₂-alkenylthio, substituted C₂-C₁₂-alkenylthio, C₂-C₁₂-alkynylthio, substituted C₂-C₁₂-alkynylthio, C₃-C₁₂ cycloalkyl and substituted C₃-C₁₂-cycloalkyl.

In a preferred embodiment, the invention relates to a compound of Formula I wherein R₂ is selected from hydrogen, halogen, C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₅-C₁₂ aryl, substituted C₅-C₁₂ aryl C₃-C₁₂ cycloalkyl and substituted C₃-C₁₂-cycloalkyl.

In a preferred embodiment, R₃ is -G₁-X₃;
wherein G₁ is absent, C₁-C₁₂-alkyl, substituted C₁-C₁₂-alkyl, C₂-C₁₂-alkenyl, substituted C₂-C₁₂-alkenyl, C₂-C₁₂-alkynyl or substituted C₂-C₁₂-alkynyl; and, X₃ is —NR₁₂R₁₃, —OR₁₂, —SR₁₂, —C(O)R₁₂, —C(O)OR₁₂, —C(O)NR₁₂R₁₃₅—N(R₁₂)C(O)R₁₃, —S(O)R₁₂ or S(O)₂R₁₂;
   wherein each R₁₂ and R₁₃ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₂ and R₁₃ together with the atoms to which they are attached and any intervening atoms may form an additional optionally substituted, 3, 4, 5, 6 or 7 membered ring.

In a preferred embodiment, the invention relates to a compound selected from Table 1:

TABLE 1

| Com- | |
|---|---|
| pound | |
| No. | |
| 1. | 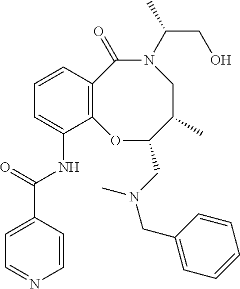 [SMILES: C[C@H](CO)N1C[C@H](C)[C@H](CN(C)Cc2ccccc2)Oc2c(NC(=O)c3ccncc3)cccc2C1=O] |
| 2. | 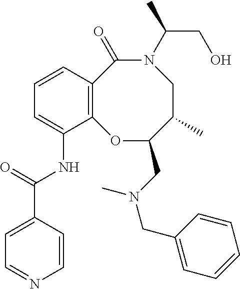 [SMILES: C[C@H]1CN([C@@H](C)CO)C(=O)c2cccc(NC(=O)c3ccncc3)c2O[C@@H]1CN(C)Cc1ccccc1] |
| 3. | 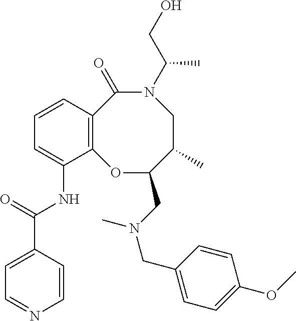 [SMILES: COc1ccc(CN(C)C[C@H]2Oc3c(NC(=O)c4ccncc4)cccc3C(=O)N([C@@H](C)CO)C[C@@H]2C)cc1] |
| 4. | 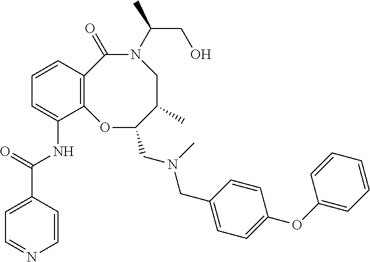 [SMILES: C[C@H]1CN([C@@H](C)CO)C(=O)c2cccc(NC(=O)c3ccncc3)c2O[C@H]1CN(C)Cc1ccc(Oc2ccccc2)cc1] |
| 5. | 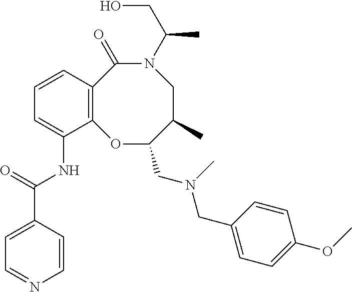 [SMILES: COc1ccc(CN(C)C[C@@H]2Oc3c(NC(=O)c4ccncc4)cccc3C(=O)N([C@H](C)CO)C[C@H]2C)cc1] |
| 6. | 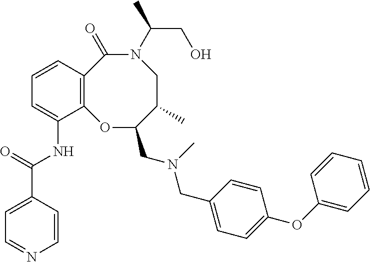 [SMILES: C[C@H]1CN([C@@H](C)CO)C(=O)c2cccc(NC(=O)c3ccncc3)c2O[C@@H]1CN(C)Cc1ccc(Oc2ccccc2)cc1] |
| 7. | 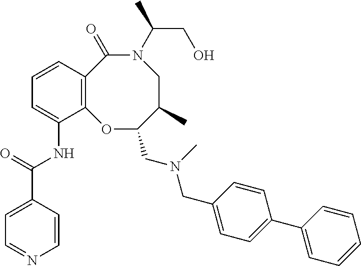 [SMILES: C[C@@H]1CN([C@@H](C)CO)C(=O)c2cccc(NC(=O)c3ccncc3)c2O[C@H]1CN(C)Cc1ccc(-c2ccccc2)cc1] |
| 8. | 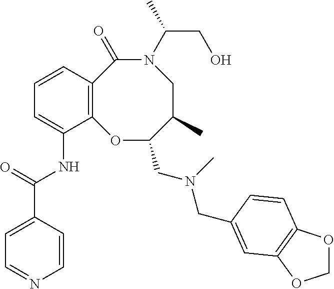 [SMILES: C[C@@H]1CN([C@H](C)CO)C(=O)c2cccc(NC(=O)c3ccncc3)c2O[C@H]1CN(C)Cc1ccc2c(c1)OCO2] |
| 9. | 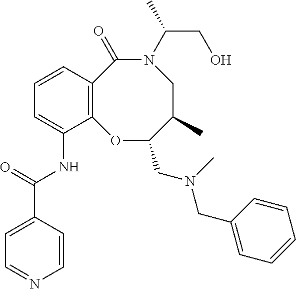 [SMILES: C[C@@H]1CN([C@H](C)CO)C(=O)c2cccc(NC(=O)c3ccncc3)c2O[C@H]1CN(C)Cc1ccccc1] |
| 10. | 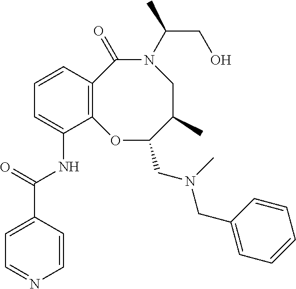 [SMILES: C[C@@H]1CN([C@@H](C)CO)C(=O)c2cccc(NC(=O)c3ccncc3)c2O[C@H]1CN(C)Cc1ccccc1] |
| 11. | 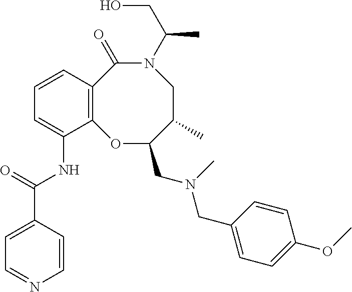 [SMILES: COc1ccc(CN(C)C[C@H]2Oc3c(NC(=O)c4ccncc4)cccc3C(=O)N([C@H](C)CO)C[C@@H]2C)cc1] |
| 12. | 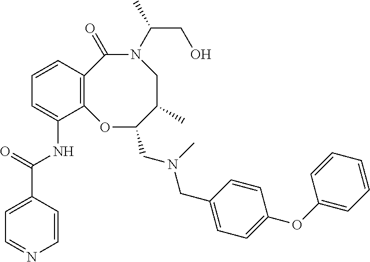 [SMILES: C[C@H](CO)N1C[C@H](C)[C@H](CN(C)Cc2ccc(Oc3ccccc3)cc2)Oc2c(NC(=O)c3ccncc3)cccc2C1=O] |
| 13. | 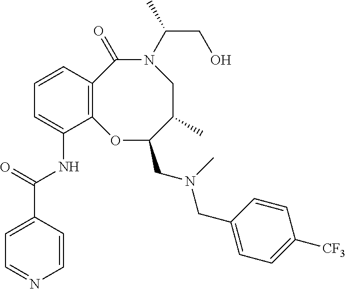 [SMILES: C[C@H](CO)N1C[C@H](C)[C@@H](CN(C)Cc2ccc(C(F)(F)F)cc2)Oc2c(NC(=O)c3ccncc3)cccc2C1=O] |
| 14. | 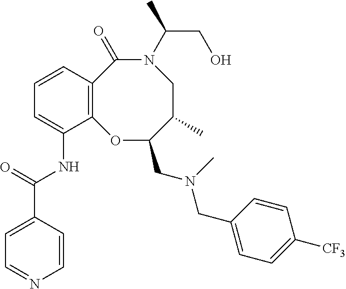 [SMILES: C[C@H]1CN([C@@H](C)CO)C(=O)c2cccc(NC(=O)c3ccncc3)c2O[C@@H]1CN(C)Cc1ccc(C(F)(F)F)cc1] |
| 15. | 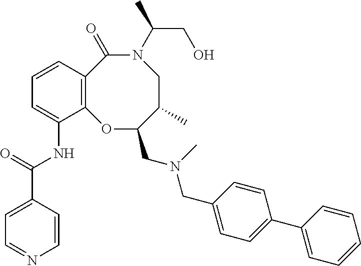 [SMILES: C[C@H]1CN([C@@H](C)CO)C(=O)c2cccc(NC(=O)c3ccncc3)c2O[C@@H]1CN(C)Cc1ccc(-c2ccccc2)cc1] |
| 16. | 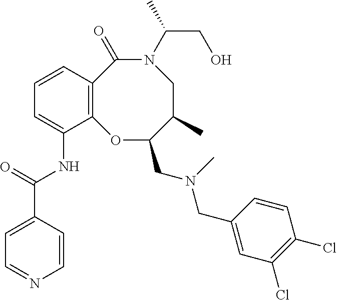 [SMILES: C[C@@H]1CN([C@H](C)CO)C(=O)c2cccc(NC(=O)c3ccncc3)c2O[C@@H]1CN(C)Cc1ccc(Cl)c(Cl)c1] |
| 17. | 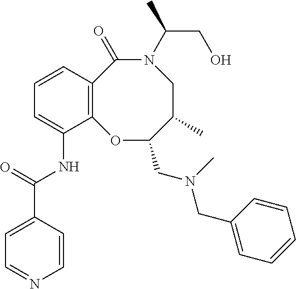 [SMILES: C[C@H]1CN([C@@H](C)CO)C(=O)c2cccc(NC(=O)c3ccncc3)c2O[C@H]1CN(C)Cc1ccccc1] |
| 18. | 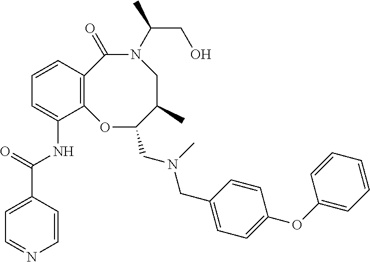 [SMILES: C[C@@H]1CN([C@@H](C)CO)C(=O)c2cccc(NC(=O)c3ccncc3)c2O[C@H]1CN(C)Cc1ccc(Oc2ccccc2)cc1] |
| 19. | 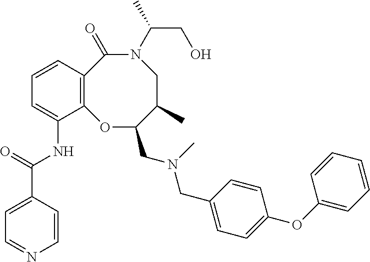 [SMILES: C[C@@H]1CN([C@H](C)CO)C(=O)c2cccc(NC(=O)c3ccncc3)c2O[C@@H]1CN(C)Cc1ccc(Oc2ccccc2)cc1] |
| 20. | 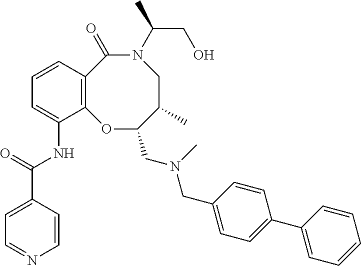 [SMILES: C[C@H]1CN([C@@H](C)CO)C(=O)c2cccc(NC(=O)c3ccncc3)c2O[C@H]1CN(C)Cc1ccc(-c2ccccc2)cc1] |

In a preferred embodiment, the invention relates to a compound selected from Table 2:

TABLE 2

| Compound | | IC₅₀ (μM) |
|---|---|---|
| No. | | (T. cruzi) |
| 21. | 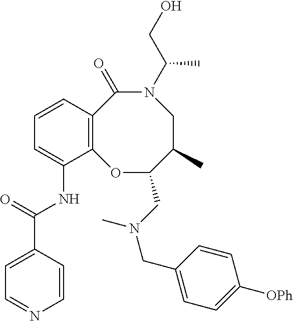 [SMILES: C[C@@H]1CN([C@@H](C)CO)C(=O)c2cccc(NC(=O)c3ccncc3)c2O[C@H]1CN(C)Cc1ccc(Oc2ccccc2)cc1] | <0.0026 |
| 22. | 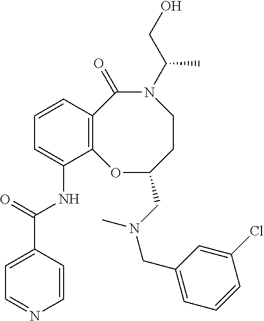 [SMILES: C[C@@H](CO)N1CC[C@H](CN(C)Cc2cccc(Cl)c2)Oc2c(NC(=O)c3ccncc3)cccc2C1=O] | <0.0026 |
| 23. | 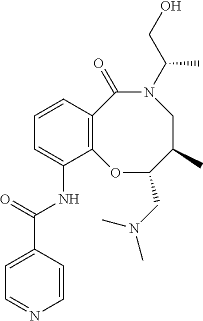 [SMILES: C[C@@H]1CN([C@@H](C)CO)C(=O)c2cccc(NC(=O)c3ccncc3)c2O[C@H]1CN(C)C] | >10 |
| 24. | 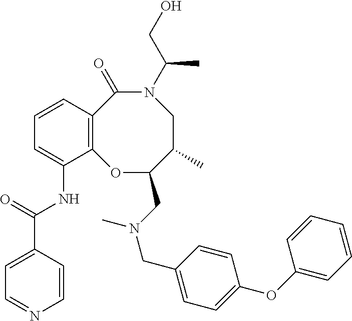 [SMILES: C[C@H](CO)N1C[C@H](C)[C@@H](CN(C)Cc2ccc(Oc3ccccc3)cc2)Oc2c(NC(=O)c3ccncc3)cccc2C1=O] | <0.0026 |
| 25. | 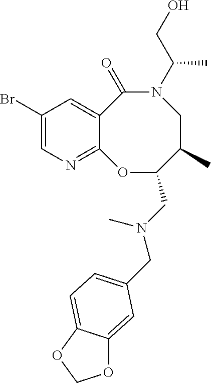 [SMILES: C[C@@H]1CN([C@@H](C)CO)C(=O)c2cc(Br)cnc2O[C@H]1CN(C)Cc1ccc2c(c1)OCO2] | >10 |
| 26. | 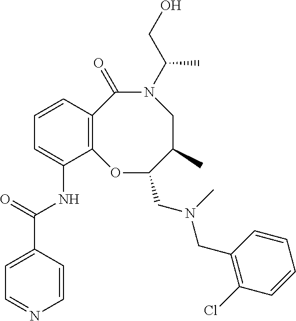 [SMILES: C[C@@H]1CN([C@@H](C)CO)C(=O)c2cccc(NC(=O)c3ccncc3)c2O[C@H]1CN(C)Cc1ccccc1Cl] | 0.0039 |
| 27. | 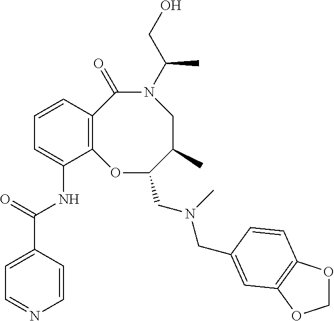 [SMILES: C[C@@H]1CN([C@H](C)CO)C(=O)c2cccc(NC(=O)c3ccncc3)c2O[C@H]1CN(C)Cc1ccc2c(c1)OCO2] | 0.00148 |
| 28. | 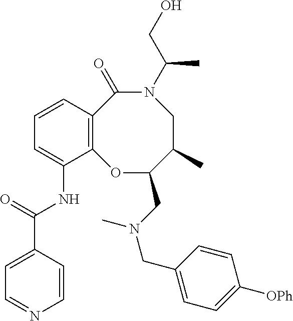 [SMILES: C[C@@H]1CN([C@H](C)CO)C(=O)c2cccc(NC(=O)c3ccncc3)c2O[C@@H]1CN(C)Cc1ccc(Oc2ccccc2)cc1] | 0.0426 |
| 29. | 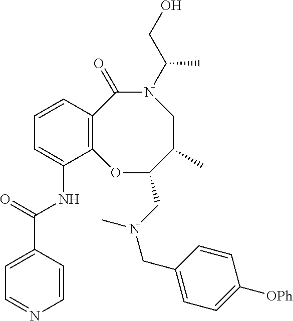 [SMILES: C[C@H]1CN([C@@H](C)CO)C(=O)c2cccc(NC(=O)c3ccncc3)c2O[C@H]1CN(C)Cc1ccc(Oc2ccccc2)cc1] | <0.0026 |
| 30. | 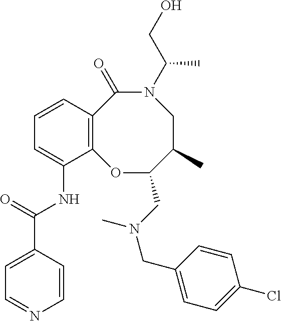 [SMILES: C[C@@H]1CN([C@@H](C)CO)C(=O)c2cccc(NC(=O)c3ccncc3)c2O[C@H]1CN(C)Cc1ccc(Cl)cc1] | <0.0026 |
| 31. | 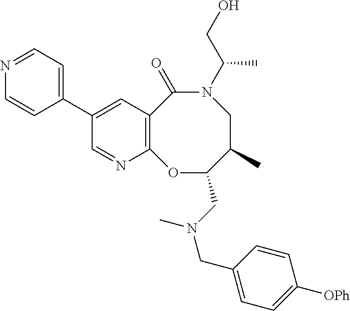 [SMILES: C[C@@H]1CN([C@@H](C)CO)C(=O)c2cc(-c3ccncc3)cnc2O[C@H]1CN(C)Cc1ccc(Oc2ccccc2)cc1] | 1.28 |
| 32. | 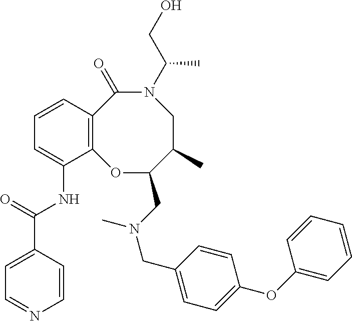 [SMILES: C[C@@H]1CN([C@@H](C)CO)C(=O)c2cccc(NC(=O)c3ccncc3)c2O[C@@H]1CN(C)Cc1ccc(Oc2ccccc2)cc1] | 0.00434 |
| 33. | 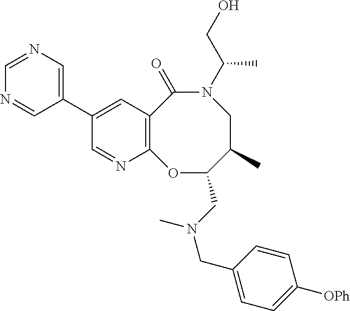 [SMILES: C[C@@H]1CN([C@@H](C)CO)C(=O)c2cc(-c3cncnc3)cnc2O[C@H]1CN(C)Cc1ccc(Oc2ccccc2)cc1] | >10 |
| 34. | 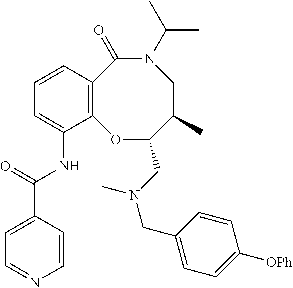 [SMILES: CC(C)N1C[C@@H](C)[C@H](CN(C)Cc2ccc(Oc3ccccc3)cc2)Oc2c(NC(=O)c3ccncc3)cccc2C1=O] | <0.0026 |
| 35. | 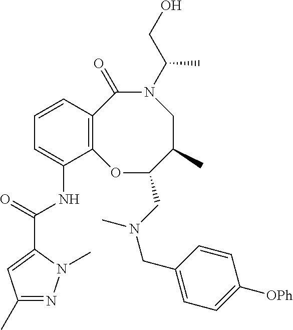 [SMILES: Cc1cc(C(=O)Nc2cccc3c2O[C@@H](CN(C)Cc2ccc(Oc4ccccc4)cc2)[C@H](C)CN([C@@H](C)CO)C3=O)n(C)n1] | >10 |
| 36. | 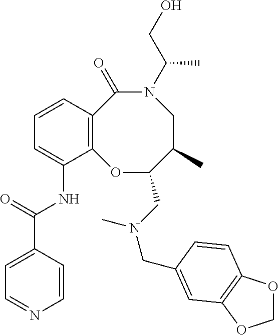 [SMILES: C[C@@H]1CN([C@@H](C)CO)C(=O)c2cccc(NC(=O)c3ccncc3)c2O[C@H]1CN(C)Cc1ccc2c(c1)OCO2] | 0.00659 |
| 37. | 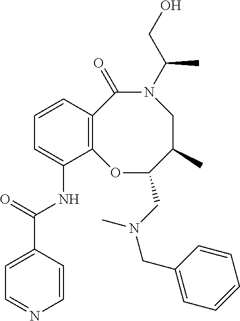 [SMILES: C[C@@H]1CN([C@H](C)CO)C(=O)c2cccc(NC(=O)c3ccncc3)c2O[C@H]1CN(C)Cc1ccccc1] | 0.0153 |
| 38. | 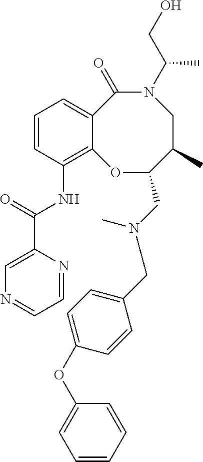 [SMILES: C[C@@H]1CN([C@@H](C)CO)C(=O)c2cccc(NC(=O)c3cnccn3)c2O[C@H]1CN(C)Cc1ccc(Oc2ccccc2)cc1] | 1 |
| 39. | 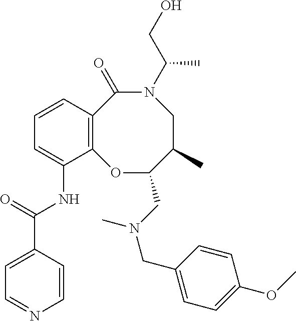 [SMILES: COc1ccc(CN(C)C[C@@H]2Oc3c(NC(=O)c4ccncc4)cccc3C(=O)N([C@@H](C)CO)C[C@H]2C)cc1] | 0.011 |
| 40. | 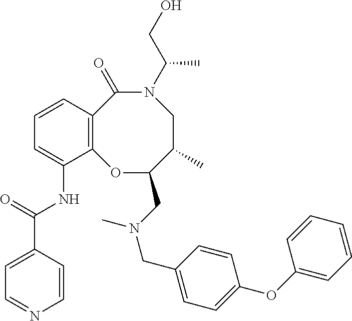 [SMILES: C[C@H]1CN([C@@H](C)CO)C(=O)c2cccc(NC(=O)c3ccncc3)c2O[C@@H]1CN(C)Cc1ccc(Oc2ccccc2)cc1] | <0.0026 |
| 41. | 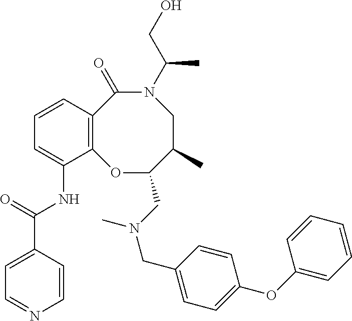 [SMILES: C[C@@H]1CN([C@H](C)CO)C(=O)c2cccc(NC(=O)c3ccncc3)c2O[C@H]1CN(C)Cc1ccc(Oc2ccccc2)cc1] | 0.000529 |
| 42. | 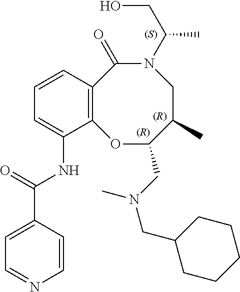 | 0.0421 |
| 43. | 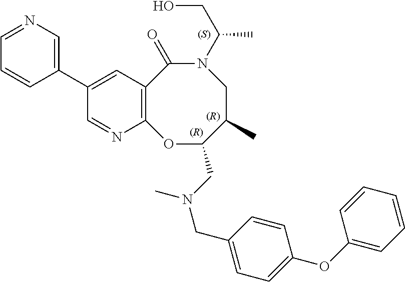 | 9.27 |

In one embodiment, the invention further provides for the use of one or more compounds of the invention in the manufacture of a medicament for halting or decreasing diseases involving parasitic infections, in particular diseases or disorders related to Trypanosomatids. In one embodiment, the invention relates to a method of treating parasitic infection in a subject in need of treatment comprising administering to said subject a therapeutically effective amount of a compound of the invention.

### DEFINITIONS

Listed below are definitions of various terms used to describe this invention. These definitions apply to the terms as they are used throughout this specification and claims, unless otherwise limited in specific instances, either individually or as part of a larger group.

The term “aliphatic group” or “aliphatic” refers to a non-aromatic moiety that may be saturated (e.g., single bond) or contain one or more units of unsaturation, e.g., double and/or triple bonds. An aliphatic group may be straight chained, branched or cyclic, contain carbon, hydrogen or, optionally, one or more heteroatoms and may be substituted or unsubstituted. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may include alkyl, substituted alkyl, alkenyl, substituted alkenyl, alkynyl, substituted alkynyl, and substituted or unsubstituted cycloalkyl groups as described herein.

The term “acyl” refers to a carbonyl substituted with hydrogen, alkyl, partially saturated or fully saturated cycloalkyl, partially saturated or fully saturated heterocycle, aryl, or heteroaryl. For example, acyl includes groups such as (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “alkyl” is intended to include both branched and straight chain, substituted or unsubstituted saturated aliphatic hydrocarbon radicals/groups having the specified number of carbons. Preferred alkyl groups comprise about 1 to about 24 carbon atoms (“C₁-C₂₄”). Other preferred alkyl groups comprise at about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The term “alkenyl” refers to linear or branched radicals having at least one carbon-carbon double bond. Such radicals preferably contain from about two to about twenty-four carbon atoms (“C₂-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkylene,” as used herein, refers to a divalent group derived from a straight chain or branched saturated hydrocarbon chain having the specified number of carbons atoms. Examples of alkylene groups include, but are not limited to, ethylene, propylene, butylene, 3-methyl-pentylene, and 5-ethyl-hexylene.

The term “alkenylene,” as used herein, denotes a divalent group derived from a straight chain or branched hydrocarbon moiety containing the specified number of carbon atoms having at least one carbon-carbon double bond. Alkenylene groups include, but are not limited to, for example, ethenylene, 2-propenylene, 2-butenylene, 1-methyl-2-buten-1-ylene, and the like.

The term “alkynylene,” as used herein, denotes a divalent group derived from a straight chain or branched hydrocarbon moiety containing the specified number of carbon atoms having at least one carbon-carbon triple bond. Representative alkynylene groups include, but are not limited to, for example, propynylene, 1-butynylene, 2-methyl-3-hexynylene, and the like.

The term “alkoxy” refers to linear or branched oxy-containing radicals each having alkyl portions of one to about twenty-four carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” refers to alkyl radicals having one or more alkoxy radicals attached to the alkyl radical, that is, to form monoalkoxyalkyl and dialkoxyalkyl radicals.

The term “aryl”, alone or in combination, means an aromatic system containing one, two or three rings wherein such rings may be attached together in a pendent manner or may be fused. The term “aryl” embraces aromatic radicals such as phenyl, naphthyl, tetrahydronaphthyl, indane furanyl, quinazolinyl, pyridyl and biphenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g., pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g., morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” refers to unsaturated aromatic heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g., 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g., benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocycloalkyl” refers to heterocyclo-substituted alkyl radicals. More preferred heterocycloalkyl radicals are “lower heterocycloalkyl” radicals having one to six carbon atoms in the heterocyclo radical.

The term “alkylthio” refers to radicals containing a linear or branched alkyl radical, of one to about ten carbon atoms attached to a divalent sulfur atom. Preferred alkylthio radicals have alkyl radicals of one to about twenty-four carbon atoms or, preferably, one to about twelve carbon atoms. More preferred alkylthio radicals have alkyl radicals which are “lower alkylthio” radicals having one to about ten carbon atoms. Most preferred are alkylthio radicals having lower alkyl radicals of one to about eight carbon atoms. Examples of such lower alkylthio radicals include methylthio, ethylthio, propylthio, butylthio and hexylthio.

The terms “aralkyl” or “arylalkyl” refer to aryl-substituted alkyl radicals such as benzyl, diphenylmethyl, triphenylmethyl, phenylethyl, and diphenylethyl.

The term “aryloxy” refers to aryl radicals attached through an oxygen atom to other radicals.

The terms “aralkoxy” or “arylalkoxy” refer to aralkyl radicals attached through an oxygen atom to other radicals.

The term “aminoalkyl” refers to alkyl radicals substituted with amino radicals. Preferred aminoalkyl radicals have alkyl radicals having about one to about twenty-four carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are “lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N,N-alkylamino, such as N-methylamino, N-ethylamino, N,N-dimethylamino, N,N-diethylamino or the like.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g., CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The terms “halogen” or “halo” as used herein, refers to an atom selected from fluorine, chlorine, bromine and iodine.

The terms “compound” “drug”, and “prodrug” as used herein all include pharmaceutically acceptable salts, co-crystals, solvates, hydrates, polymorphs, enantiomers, diastereoisomers, racemates and the like of the compounds, drugs and prodrugs having the formulas as set forth herein.

Substituents indicated as attached through variable points of attachments can be attached to any available position on the ring structure.

As used herein, the term “effective amount of the subject compounds,” with respect to the subject method of treatment, refers to an amount of the subject compound which, when delivered as part of desired dose regimen, brings about management of the disease or disorder to clinically acceptable standards.

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., infection of others) of disease, and amelioration of the disease state (whether partial or total).

“Combination therapy” includes the administration of the subject compounds in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, preferably compounds that are able to enhance the effect of the compounds of the invention. The compounds of the invention can be administered simultaneously (as a single preparation or separate preparation) or sequentially to the other drug therapy. In general, a combination therapy envisions administration of two or more drugs during a single cycle or course of therapy.

Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid, gel or liquid filler, diluent, encapsulating material or formulation auxiliary of any type. Some examples of materials which can serve as pharmaceutically acceptable carriers are sugars such as lactose, glucose and sucrose; cyclodextrins such as alpha- (α), beta- (β) and gamma- (γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir. In a preferred embodiment, administration is parenteral administration by injection.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable suspension or emulsion, such as INTRALIPID®, LIPOSYN® or OMEGAVEN®, or solution, in a nontoxic parenterally acceptable diluent or solvent, for example, as a solution in 1,3-butanediol. INTRALIPID® is an intravenous fat emulsion containing 10-30% soybean oil, 1-10% egg yolk phospholipids, 1-10% glycerin and water. LIPOSYN® is also an intravenous fat emulsion containing 2-15% safflower oil, 2-15% soybean oil, 0.5-5% egg phosphatides 1-10% glycerin and water. OMEGAVEN® is an emulsion for infusion containing about 5-25% fish oil, 0.5-10% egg phosphatides, 1-10% glycerin and water. Among the acceptable vehicles and solvents that may be employed are water, Ringer's solution, USP and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics is known in the art (see, for example U.S. Pat. No. 5,767,068 to VanDevanter et al., U.S. Pat. No. 5,508,269 to Smith et al., and WO 98/43650 by Montgomery).

### EXAMPLES

### Synthesis

General methods of synthesizing compounds of Formulas I, IA, II, III, IV and V are described in the Scheme and general procedures set forth below. A synthetic protocol for the synthesis of macrocyclic compounds is described in Marcaurelle L. A. et al., J. Am. Chem. Soc. 132, 16962-16976, 2010.

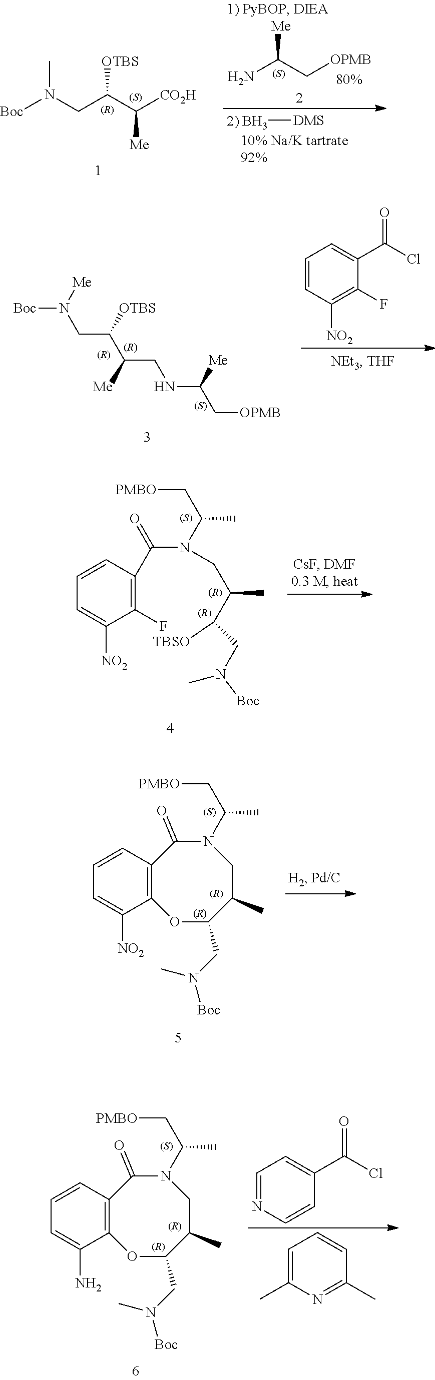

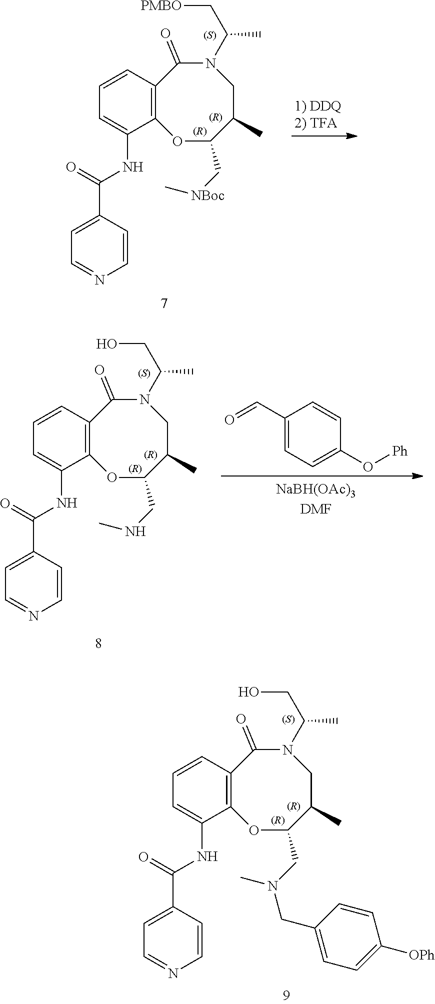

### Chemistry Experimental Methods

### General.

All oxygen and/or moisture sensitive reactions were carried out under N₂ atmosphere in glassware that had been flame-dried under vacuum (˜0.5 mm Hg) and purged with N₂ prior to use. All reagents and solvents were purchased from commercial vendors and used as received, or synthesized according to the footnoted references. NMR spectra were recorded on a Bruker 300 (300 MHz ¹H, 75 MHz ¹³C) or Varian UNITY INOVA 500 (500 MHz ¹H, 125 MHz ¹³C) spectrometer. Proton and carbon chemical shifts are reported in ppm (δ) referenced to the NMR solvent. Data are reported as follows: chemical shifts, multiplicity (br=broad, s=singlet, d=doublet, t=triplet, q=quartet, m=multiplet; coupling constant(s) in Hz). Unless otherwise indicated NMR data were collected at 25° C. Flash chromatography was performed using 40-60 μm Silica Gel (60 Å mesh) on a Teledyne Isco Combiflash R_{f}. Tandem Liquid Chromotography/Mass Spectrometry (LCMS) was performed on a Waters 2795 separations module and 3100 mass detector. Analytical thin layer chromatography (TLC) was performed on EM Reagent 0.25 mm silica gel 60-F plates. Visualization was accomplished with UV light and aqueous potassium permanganate (KMnO₄) stain followed by heating. High-resolution mass spectra were obtained at the MIT Mass Spectrometry Facility (Bruker Daltonics APEXIV 4.7 Tesla Fourier Transform Ion Cyclotron Resonance Mass Spectrometer).

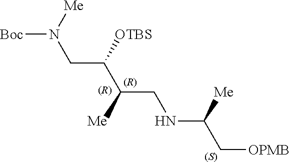

### tert-Butyl 2-(tert-butyldimethylsilyloxy)-4-(1-(4-methoxybenzyloxy)propan-2-ylamino)-3-methylbutylmethyl)carbamate, 3

An oven-dried, 3-L, 3-neck round bottom flask was equipped with an overhead stirrer, addition funnel and a temperature probe. Under a positive flow of N₂, the vessel was charged with 4-(tert-butoxycarbonyl(methyl)amino)-3-(tert-butyldimethylsilyloxy)-2-methylbutanoic acid 1 (1.0 equiv) dissolved in CH₂Cl₂ (80% of total solvent, final concentration of 1=0.2 M), followed by PyBOP (1.0 equiv), and diisopropyl ethylamine (DIPEA) (3.0 equiv). The resulting mixture was cooled in an ice bath before 1-(4-methoxybenzyloxy)propan-2-amine 2 (1.1-1.2 equiv) was added as a solution in CH₂Cl₂ (remaining 20% of total solvent) by addition funnel. The rate of addition was controlled so as to maintain an internal temperature between 3-5° C. When addition was complete, the mixture was warmed to ambient temperature and allowed to stir for 15 h. The reaction was quenched with water, and extracted with CH₂Cl₂. The combined organic extracts were dried over MgSO₄, filtered and concentrated. The yellow oil was taken up in Et₂O, and the phosphoramide byproducts were removed via filtration. The solvent was removed in vacuo, and the crude product was isolated. Flash chromatography on silica gel (4:1 Hexanes/EtOAc to 7:3 Hexanes/EtOAc) gave the product a colorless oil.

An oven-dried, 2-L, 1-necked round bottom flask was equipped with a magnetic stirrer. Under a positive flow of N₂, the flask was charged with tert-butyl 2-(tert-butyldimethylsilyloxy)-4-(1-(4-methoxybenzyloxy)propan-2-ylamino)-3-methyl-4-oxobutyl(methyl)carbamate (1.0 equiv) and anhydrous THF (final concentration 0.1 M). Borane dimethylsulfide complex (BH₃DMS) (5.0 equiv) was added dropwise via syringe. Afterwards the reaction mixture was heated at 65° C. for 5 h. After cooling to ambient temperature, excess hydride was quenched by the careful addition of MeOH. The mixture was concentrated under reduced pressure to afford a colorless oil, which was then co-evaporated with MeOH three times to remove excess B(OMe)₃. The oil was then re-dissolved in MeOH and 10% aqueous potassium sodium tartrate (2:3 ratio, final concentration 0.067 M). The resulting slurry was heated at reflux for 12 h. The volatiles were removed under reduced pressure and aqueous layer was extracted three times with ethyl acetate. The combined organic layers were washed once with brine, dried over magnesium sulfate, filtered and concentrated to provide the desired amine 3 as a colorless oil. [α]_{D}²⁰ −10.65 (c 2.46, CHCl₃). ¹H NMR (500 MHz, CDCl₃, 60° C.) δ 7.19 (d, J=8.5 Hz, 2H), 6.82 (d, J=8.5 Hz, 2H), 4.40 (s, 2H), 3.89 (m, 1H), 3.74 (s, 3H), 3.29 (m, 4H), 2.98 (dd, J=13.5, 7.5 Hz, 1H), 2.85 (m, 4H), 2.63 (m, 1H), 2.41 (dd, J=11, 9 Hz, 1H), 1.72 (m, 1H), 1.41 (s, 9H), 0.98 (d, J=6.5 Hz, 1H), 0.92 (d, J=6.5 Hz, 1H), 0.86 (s, 9H), 0.02 (s, 3H), 0.00 (s, 3H). ¹³C NMR (125 MHz, CDCl₃, 60° C.) δ 159.3, 155.7, 130.8, 129.1, 113.9, 79.1, 74.6, 73.3, 72.8, 55.2, 52.5, 52.0, 49.4, 38.7, 36.4, 28.6, 26.0, 18.0, 17.6, 13.7, −4.5. HRMS (ESI) calcd for C₂₈H₅₃N₂O₅Si [M+H]⁺: 525.3718. Found: 525.3698.

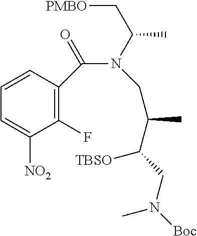

### tert-Butyl-((2R,3R)-2-((tert-butyldimethylsilyl)oxy)-4-(2-fluoro-N—((S)-1-((4-methoxybenzyl)oxy)propan-2-yl)-3-nitrobenzamido)-3-methylbutyl)(methyl)carbamate, 4

To a stirred solution of 3 (21 g, 40.0 mmol, 1 equiv) and 2-fluoro-3-nitrobenzoyl chloride (20.36 g, 100 mmol, 2.5 equiv) in CH₂Cl₂ (120 mL) at 0° C. was added NEt₃ (27.7 mL, 200 mmol, 5 equiv). The reaction was allowed to warm to room temperature as it stirred, and no starting material remained after 1.5 h. H₂O (50 mL) was added to the reaction and it was extracted with CH₂Cl₂ (2×100 mL). The combined organic portion was dried over MgSO₄, filtered, and concentrated. The crude material was purified by silica gel chromatography in EtOAc/hexanes (10%→30%) to give the product. [α]²⁰_{D} −49.5 (c 1.0, CHCl₃); ¹H NMR (DMSO-d₆, 500 MHz, 150° C.) δ 8.12 (dd, J=7.7, 7.7 Hz, 1H), 7.64 (dd, J=6.6, 6.6 Hz, 1H), 7.45 (dd, J=7.9, 7.9 Hz, 1H), 7.21 (d, J=8.4 Hz, 2H), 6.90 (d, J=8.4 Hz, 2H), 4.41 (s, 2H), 3.95-3.83 (m, 2H), 3.78 (s, 3H), 3.54-3.45 (m, 1H), 3.40-3.24 (m, 4H), 3.16-3.08 (m, 1H), 2.84 (s, 3H), 2.15-2.06 (m, 1H), 1.43 (s, 9H), 1.25 (br s, 3H), 0.94 (br s, 3H), 0.85 (s, 9H), 0.04 (s, 3H), 0.02 (s, 3H); ¹³C NMR (DMSO-d₆, 125 MHz, 150° C.) δ 164.2, 158.5, 154.5, 149.7, 137.1, 133.5 , 129.7, 128.2 (2C), 128.0, 125.3, 124.6, 113.3 (2C), 78.1, 72.4 71.5, 70.4, 54.6, 54.0, 51.0, 36.3 (br), 34.8, 27.5 (3C), 25.0 (3C), 16.9, 14.7, 12.5, −5.4, −5.6 (1 carbon absent); HRMS (ESI) calc'd for C₃₅H₅₅FN₃O₈Si [M+H]⁺: 692.3737. found: 692.3764.

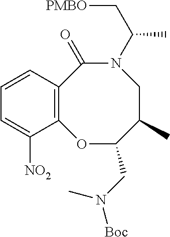

### tert-Butyl (((2R,3R)-5-((S)-1-((4-methoxybenzyl)oxy)propan-2-yl)-3-methyl-10-nitro-6-oxo-3,4,5,6-tetrahydro-2H-benzo[b][1,5]oxazocin-2-yl)methyl)(methyl)carbamate, 5

To a stirred solution of 4 (24.5 g, 35.4 mmol, 1 equiv) in DMF (708 mL) was added CsF (10.76 g, 70.8 mmol (2 equiv). The resulting suspension was heated to 85° C. for 5 h. The solvent was then removed under reduced pressure, and the crude solid was dissolved in EtOAc (250 mL), washed with H₂O (1×100 mL) and brine (1×100 mL), dried over Na₂SO₄, filtered, and concentrated. The product was used in the next reaction without purification. [α]²⁰_{D} −52.1 (c 1.0, CHCl₃); ¹H NMR (DMSO-d₆, 500 MHz, 150° C.) δ 7.91 (d, J=8.0 Hz, 1H), 7.64 (d, J=8.0 Hz, 1H), 7.39 (dd, J=8.0, 8.0 Hz, 1H), 7.27 (d, J=8.4 Hz, 2H), 6.91 (d, J=8.4 Hz, 2H), 4.52 (d, J=11.8 Hz, 1H), 4.48 (d, J=11.8 Hz, 1H), 4.35-4.28 (m, 1H), 3.96-3.91 (m, 1H), 3.86 (dd, J=7.3, 9.8 Hz, 1H), 3.79 (s, 3H), 3.66 (dd, J=5.8, 10.2 Hz, 1H), 3.63 (dd, J=5.0, 15.0 Hz, 1H), 3.50 (dd, J=2.0, 15.0 Hz, 1H), 3.38 (dd, J=10.0, 16.0 Hz, 1H), 3.15 (br d, J=16.0 Hz, 1H), 2.89 (s, 3H), 2.18-2.12 (m, 1H), 1.47 (s, 9H), 1.34 (d, J=6.7 Hz, 3H), 0.87 (d, J=6.8 Hz, 3H); ¹³C NMR (DMSO-d₆, 125 MHz, 150° C.) δ 165.0, 158.5, 154.5, 146.6, 143.0, 133.7, 132.2, 130.0, 128.0, 125.4, 124.3, 113.3, 88.5, 78.2, 71.4, 71.0, 54.6, 52.4, 51.5, 50.4, 35.2, 34.4, 27.4, 15.2, 13.9; HRMS (ESI) calcd for C₂₉H₃₉N₃NaO₈ [M+Na]⁺: 580.2629. found: 580.2614.

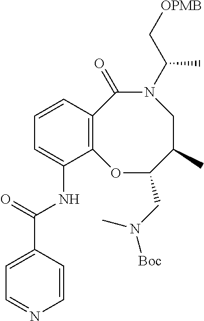

### tert-butyl (((2R,3R)-10-(isonicotinamido)-5-((S)-1-((4-methoxybenzyl)oxy)propan-2-yl)-3-methyl-6-oxo-3,4,5,6-tetrahydro-2H-benzo[b][1,5]oxazocin-2-yl)methyl)(methyl)carbamate, 7

A mixture of compound 5 and Pd/C in EtOH were stirred under H₂ at 40° C. No starting material remained after 1.5 h, and the reaction was cooled, filtered through Celite, and concentrated to give tert-butyl (((2R,3R)-10-amino-5-((S)-1-((4-methoxybenzyl)oxy)propan-2-yl)-3-methyl-6-oxo-3,4,5,6-tetrahydro-2H-benzo[b][1,5]oxazocin-2-yl)methyl)(methyl)carbamate 6. The crude material was used in the next reaction without purification. The crude material was dissolved in dichloromethante and 2,6-lutidine and isonicotinoyl chloride were added. The reaction was stirred at room temperature overnight. The reaction was concentrated, and purified by silica chromatography to give 7.

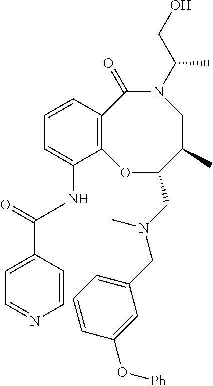 [SMILES: C[C@@H]1CN([C@@H](C)CO)C(=O)c2cccc(NC(=O)c3ccncc3)c2O[C@H]1CN(C)Cc1cccc(Oc2ccccc2)c1]

### N-((2R,3R)-5-((S)-1-hydroxypropan-2-yl)-3-methyl-2-((methyl(3-phenoxybenzyl)amino)methyl)-6-oxo-3,4,5,6-tetrahydro-2H-benzo[b][1,5]oxazocin-10-yl)isonicotinamide, 9

Compound 7 was dissolved in dichloromethane and pH 7 buffer was added. The mixture was cooled to 0° C. and 2,3-Dichloro-5,6-dicyano-1,4-benzoquinone was added. The reaction was allowed to stir overnight as it warmed to room temperature. The reaction was cooled to 0° C., quenched with H2O, and the aqueous portion was extracted with dichloromethane two times. The organic portion was washed with saturated sodium bicarbonate and then activated carbon was added. It was filtered through celite and the celite pad was washed with hot dichloromethane. The solvent was removed under reduced pressure and the crude material was purified by silica chromatography to yield tert-butyl ((2R,3R)-5-((S)-1-hydroxypropan-2-yl)-10-(isonicotinamido)-3-methyl-6-oxo-3,4,5,6-tetrahydro-2H-benzo[b][1,5]oxazocin-2-yl)methyl(methyl)carbamate. tert-butyl ((2R,3R)-5-((S)-1-hydroxypropan-2-yl)-10-(isonicotinamido)-3-methyl-6-oxo-3,4,5,6-tetrahydro-2H-benzo[b][1,5]oxazocin-2-yl)methyl(methyl)carbamate was dissolved in dichloromethane and trifluoroacetic acid was added. The reaction was stirred at room temperature. The reaction was quenched with sodium bicarbonate and extracted with ethyl acetate. Dried with magnesium sulfate, filtered and concentrated under vacuum. The crude material was used directly in the next step. The crude material was dissolved in dimethylformamide with catalytic acetic acid. 4-phenoxybenzaldehyde and sodium triacetoxyborohydride were added. The reaction was stirred at room temperature overnight. Added potassium carbonate to neutralize the acid, then removed dimethylformamide in vacuo, added water and EtOAc, and stirred for 30 min. Extracted with ethyl acetate, dried over sodium sulfate and concentrated. The product was purified by silica chromatography. ESI [M+H]=595.30.

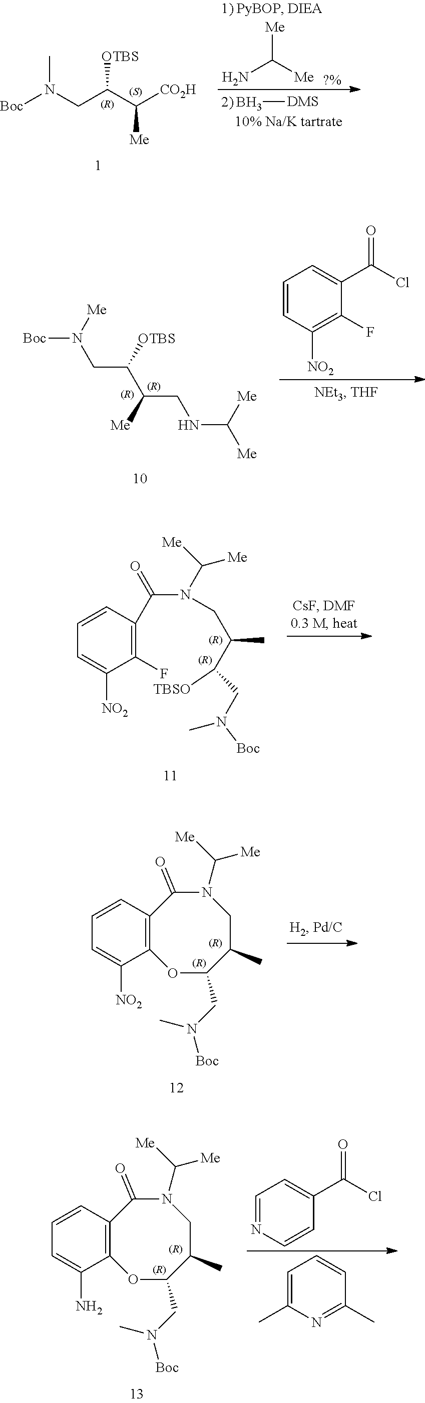

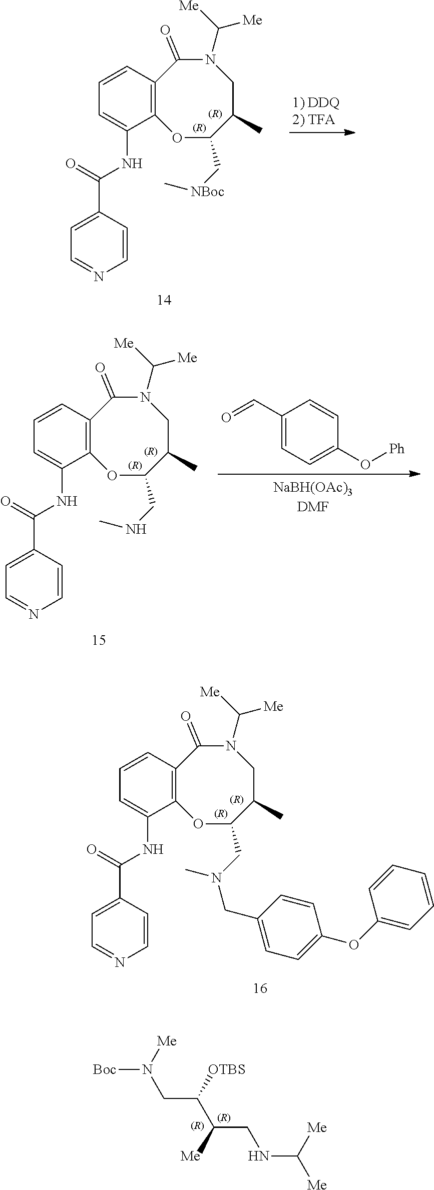

### tert-butyl ((2R,3R)-2-((tert-butyldimethylsilyl)oxy)-4-(isopropylamino)-3-methylbutyl)(methyl)carbamate, 10

An oven-dried, 3-L, 3-neck round bottom flask was equipped with an overhead stirrer, addition funnel and a temperature probe. Under a positive flow of N₂, the vessel was charged with 4-(tert-butoxycarbonyl(methyl)amino)-3-(tert-butyldimethylsilyloxy)-2-methylbutanoic acid 1 (1.0 equiv) dissolved in CH₂Cl₂ (80% of total solvent, final concentration of 1=0.2 M), followed by PyBOP (1.0 equiv), and diisopropyl ethylamine (DIPEA) (3.0 equiv). The resulting mixture was cooled in an ice bath before isopropyl amine (1.1-1.2 equiv) was added as a solution in CH₂Cl₂ (remaining 20% of total solvent) by addition funnel. The rate of addition was controlled so as to maintain an internal temperature between 3-5° C. When addition was complete, the mixture was warmed to ambient temperature and allowed to stir for 15 h. The reaction was quenched with water, and extracted with CH₂Cl₂. The combined organic extracts were dried over MgSO₄, filtered and concentrated. The yellow oil was taken up in Et₂O, and the phosphoramide byproducts were removed via filtration. The solvent was removed in vacuo, and the crude product was isolated. Flash chromatography on silica gel (4:1 Hexanes/EtOAc to 7:3 Hexanes/EtOAc) gave tert-butyl ((2R,3S)-2-((tert-butyldimethylsilyl)oxy)-4-(isopropylamino)-3-methyl-4-oxobutyl)(methyl)carbamate as a colorless oil.

An oven-dried, 2-L, 1-necked round bottom flask was equipped with a magnetic stirrer. Under a positive flow of N₂, the flask was charged with tert-butyl ((2R,3S)-2-((tert-butyldimethylsilyl)oxy)-4-(isopropylamino)-3-methyl-4-oxobutyl)(methyl)carbamate (1.0 equiv) and anhydrous THF (final concentration 0.1 M). Borane dimethylsulfide complex (BH₃.DMS) (5.0 equiv) was added dropwise via syringe. Afterwards the reaction mixture was heated at 65° C. for 5 h. After cooling to ambient temperature, excess hydride was quenched by the careful addition of MeOH. The mixture was concentrated under reduced pressure to afford a colorless oil, which was then co-evaporated with MeOH three times to remove excess B(OMe)₃. The oil was then re-dissolved in MeOH and 10% aqueous potassium sodium tartrate (2:3 ratio, final concentration 0.067 M). The resulting slurry was heated at reflux for 12 h. The volatiles were removed under reduced pressure and aqueous layer was extracted three times with ethyl acetate. The combined organic layers were washed once with brine, dried over magnesium sulfate, filtered and concentrated to provide the desired amine 9 as a colorless oil.

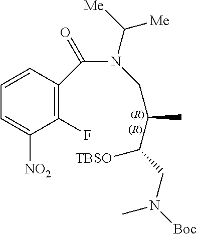

### tert-butyl ((2R,3R)-2-((tert-butyldimethylsilyl)oxy)-4-(2-fluoro-N-isopropyl-3-nitrobenzamido)-3-methylbutyl)(methyl)carbamate, 11

To a stirred solution of 10 (21 g, 40.0 mmol, 1 equiv) and 2-fluoro-3-nitrobenzoyl chloride (20.36 g, 100 mmol, 2.5 equiv) in CH₂Cl₂ (120 mL) at 0° C. was added NEt₃ (27.7 mL, 200 mmol, 5 equiv). The reaction was allowed to warm to room temperature as it stirred, and no starting material remained after 1.5 h. H₂O (50 mL) was added to the reaction and it was extracted with CH₂Cl₂ (2×100 mL). The combined organic portion was dried over MgSO₄, filtered, and concentrated. The crude material was purified by silica gel chromatography in EtOAc/hexanes (10%→30%) to give the product.

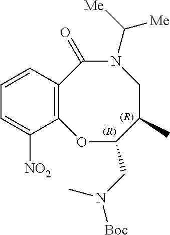

### tert-butyl (((2R,3R)-5-isopropyl-3-methyl-10-nitro-6-oxo-3,4,5,6-tetrahydro-2H-benzo[b][1,5]oxazocin-2-yl)methyl)(methyl)carbamate, 12

To a stirred solution of 11 (24.5 g, 35.4 mmol, 1 equiv) in DMF (708 mL) was added CsF (10.76 g, 70.8 mmol (2 equiv). The resulting suspension was heated to 85° C. for 5 h. The solvent was then removed under reduced pressure, and the crude solid was dissolved in EtOAc (250 mL), washed with H₂O (1×100 mL) and brine (1×100 mL), dried over Na₂SO₄, filtered, and concentrated. The product was used in the next reaction without purification.

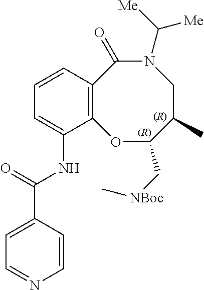

### tert-butyl (((2R,3R)-10-(isonicotinamido)-5-isopropyl-3-methyl-6-oxo-3,4,5,6-tetrahydro-2H-benzo[b][1,5]oxazocin-2-yl)methyl)(methyl)carbamate, 14

A mixture of compound 12 and Pd/C in EtOH were stirred under H₂ at 40° C. No starting material remained after 1.5 h, and the reaction was cooled, filtered through Celite, and concentrated to give tert-butyl (((2R,3R)-10-amino-5-isopropyl-3-methyl-6-oxo-3,4,5,6-tetrahydro-2H-benzo[b][1,5]oxazocin-2-yl)methyl)(methyl)carbamate 13. The crude material was used in the next reaction without purification. The crude material was dissolved in dichloromethante and 2,6-lutidine and isonicotinoyl chloride were added. The reaction was stirred at room temperature overnight. The reaction was concentrated, and purified by silica chromatography.

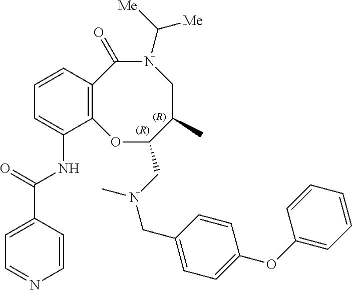

### N-((2R,3R)-5-isopropyl-3-methyl-2-((methyl(4-phenoxybenzyl)amino)methyl)-6-oxo-3,4,5,6-tetrahydro-2H-benzo[b][1,5]oxazocin-10-yl)isonicotinamide, 16

Compound 14 was dissolved in dichloromethane and trifluoroacetic acid was added. The reaction was stirred at room temperature. The reaction was quenched with sodium bicarbonate and extracted with ethyl acetate. Dried with magnesium sulfate, filtered and concentrated under vacuum. The crude material was used directly in the next step. The crude material was dissolved in dimethylformamide with catalytic acetic acid. 4-phenoxybenzaldehyde and sodium triacetoxyborohydride were added. The reaction was stirred at room temperature overnight. Added potassium carbonate to neutralize the acid, then removed dimethylformamide in vacuo, added water and EtOAc, and stirred for 30 min. Extracted with ethyl acetate, dried over sodium sulfate and concentrated. The product was purified by silica chromatography. ESI [M+H]=579.15.

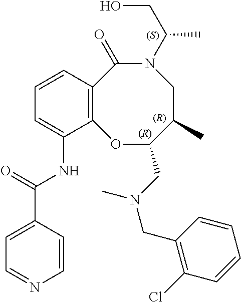

### N-((2R,3R)-2-(((2-chlorobenzyl)(methyl)amino)methyl)-5-((S)-1-hydroxypropan-2-yl)-3-methyl-6-oxo-3,4,5,6-tetrahydro-2H-benzo[b][1,5]oxazocin-10-yl)isonicotinamide, 17

Synthesized from compound 7 was using the procedure for 9 replacing 4-phenoxybenzaldehyde with 2-chlorobenzaldehyde.

ESI [M+H]=537.25.

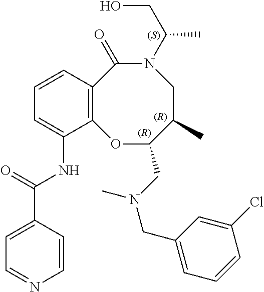

### N-((2R,3R)-2-(((3-chlorobenzyl)(methyl)amino)methyl)-5-((S)-1-hydroxypropan-2-yl)-3-methyl-6-oxo-3,4,5,6-tetrahydro-2H-benzo[b][1,5]oxazocin-10-yl)isonicotinamide, 18

Synthesized from compound 7 was using the procedure for 9 replacing 4-phenoxybenzaldehyde with 3-chlorobenzaldehyde.

ESI [M+H]=537.23.

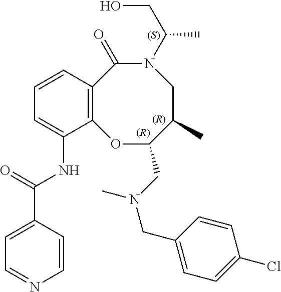

### N-((2R,3R)-2-(((4-chlorobenzyl)(methyl)amino)methyl)-5-((S)-1-hydroxypropan-2-yl)-3-methyl-6-oxo-3,4,5,6-tetrahydro-2H-benzo[b][1,5]oxazocin-10-yl)isonicotinamide, 19

Synthesized from compound 7 was using the procedure for 9 replacing 4-phenoxybenzaldehyde with 4-chlorobenzaldehyde.

ESI [M+H]=537.26.

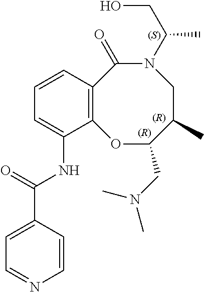

### N-((2R,3R)-2-((dimethylamino)methyl)-5-((S)-1-hydroxypropan-2-yl)-3-methyl-6-oxo-3,4,5,6-tetrahydro-2H-benzo[b][1,5]oxazocin-10-yl)isonicotinamide, 20

Synthesized from compound 7 was using the procedure for 9 replacing 4-phenoxybenzaldehyde with formaldehyde. ESI [M+]=426.2.

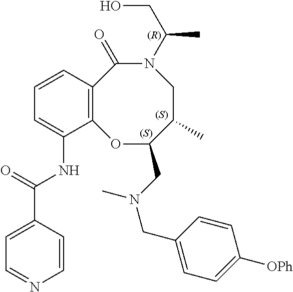

### N-((2S,3S)-5-((R)-1-hydroxypropan-2-yl)-3-methyl-2-((methyl(4-phenoxybenzyl)amino)methyl)-6-oxo-3,4,5,6-tetrahydro-2H-benzo[b][1,5]oxazocin-10-yl)isonicotinamide, 21

Synthesized using the procedure for 9 replacing 3 with the (R,S,S) amine. ESI [M+]=594.3.

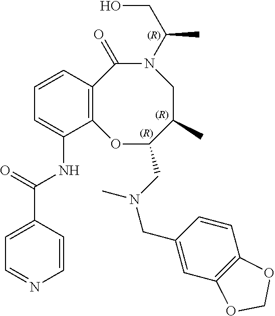

### N-((2R,3R)-2-(((benzo[d][1,3]dioxol-5-ylmethyl)(methyl)amino)methyl)-5-((R)-1-hydroxypropan-2-yl)-3-methyl-6-oxo-3,4,5,6-tetrahydro-2H-benzo[b][1,5]oxazocin-10-yl)isonicotinamide, 22

Synthesized using the procedure for 9 replacing 3 with the (R,R,R) amine and 4-phenoxybenzaldehyde with piperonaldehyde. ESI [M+H]=547.40.

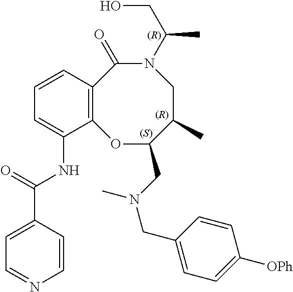

### N-((2S,3R)-5-((R)-1-hydroxypropan-2-yl)-3-methyl-2-((methyl(4-phenoxybenzyl)amino)methyl)-6-oxo-3,4,5,6-tetrahydro-2H-benzo[b][1,5]oxazocin-10-yl)isonicotinamide, 23

Synthesized using the procedure for 9 replacing 3 with the (R,R,S) amine. ESI [M+]=594.3.

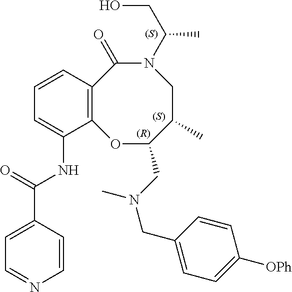

### N-((2R,3 S)-5-(R)-1-hydroxypropan-2-yl)-3-methyl-2-((methyl(4-phenoxybenzyl)amino)methyl)-6-oxo-3,4,5,6-tetrahydro-2H-benzo[b][1,5]oxazocin-10-yl)isonicotinamide, 24

Synthesized using the procedure for 9 replacing 3 with the (S,S,R) amine. ESI [M+H]=595.4.

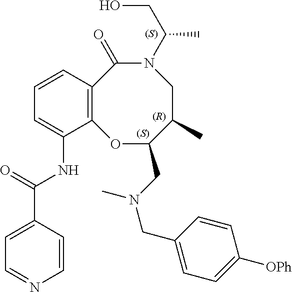

### N-((2 S,3R)-5-((S)-1-hydroxypropan-2-yl)-3-methyl-2-((methyl(4-phenoxybenzyl)amino)methyl)-6-oxo-3,4,5,6-tetrahydro-2H-benzo[b][1,5]oxazocin-10-yl)isonicotinamide, 25

Synthesized using the procedure for 9 replacing 3 with the (S,R,S) amine. ESI [M+]=594.3.

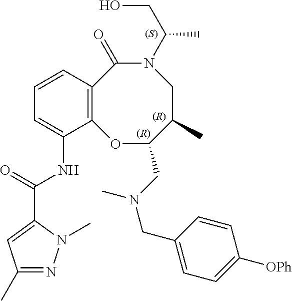

### N-((2R,3R)-5-((S)-1-hydroxypropan-2-yl)-3-methyl-2-((methyl(4-phenoxybenzyl)amino)methyl)-6-oxo-3,4,5,6-tetrahydro-2H-benzo[b][1,5]oxazocin-10-yl)-1,3-dimethyl-1H-pyrazole-5-carboxamide, 26

Synthesized using the procedure for 9 replacing isonicotynyl chloride with 1,3-dimethyl-1H-pyrazole-5-carbonyl chloride. ESI [M+H]=612.29.

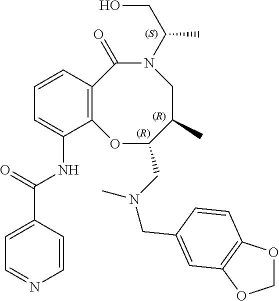

### N-((2R,3R)-2-(((benzo[d][1,3]dioxol-5-ylmethyl)(methyl)amino)methyl)-5-((S)-1-hydroxypropan-2-yl)-3-methyl-6-oxo-3,4,5,6-tetrahydro-2H-benzo[b][1,5]oxazocin-10-yl)isonicotinamide, 27

Synthesized from compound 7 was using the procedure for 9 replacing 4-phenoxybenzaldehyde with piperonaldehyde. ESI [M+H]=547.20.

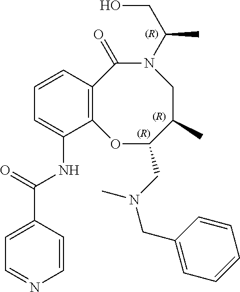

### N-((2R,3R)-2-((benzyl(methyl)amino)methyl)-5-((R)-1-hydroxypropan-2-yl)-3-methyl-6-oxo-3,4,5,6-tetrahydro-2H-benzo[b][1,5]oxazocin-10-yl)isonicotinamide, 28

Synthesized using the procedure for 9 replacing 3 with the (R,R,R) amine and 4-phenoxybenzaldehyde with benzaldehyde. ESI [M+H]=502.3.

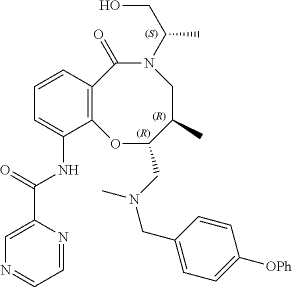

### N-((2R,3R)-5-((S)-1-hydroxypropan-2-yl)-3-methyl-2-((methyl(4-phenoxybenzyl)amino)methyl)-6-oxo-3,4,5,6-tetrahydro-2H-benzo[b][1,5]oxazocin-10-yl)pyrazine-2-carboxamide, 29

Synthesized using the procedure for 9 replacing isonicotynyl chloride with 1,3-dimethyl-1H-pyrazole-5-carbonyl chloride. ESI [M+H]=596.27.

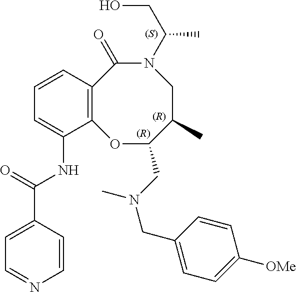

### N-((2R,3R)-5-((S)-1-hydroxypropan-2-yl)-2-(((4-methoxybenzyl)(methyl)amino)methyl)-3-methyl-6-oxo-3,4,5,6-tetrahydro-2H-benzo[b][1,5]oxazocin-10-yl)isonicotinamide, 30

Synthesized using the procedure for 9 replacing 4-phenoxybenzaldehyde with 4-methoxybenzaldehyde. [M+H]=533.09.

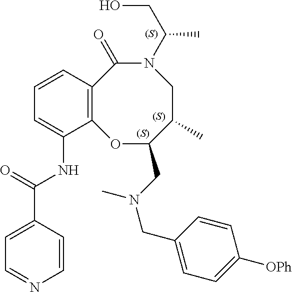

### N-((2S,3S)-5-((S)-1-hydroxypropan-2-yl)-3-methyl-2-((methyl(4-phenoxybenzyl)amino)methyl)-6-oxo-3,4,5,6-tetrahydro-2H-benzo[b][1,5]oxazocin-10-yl)isonicotinamide, 31

Synthesized using the procedure for 9 replacing 3 with the (S,S,S) amine. [M+H]=595.43.

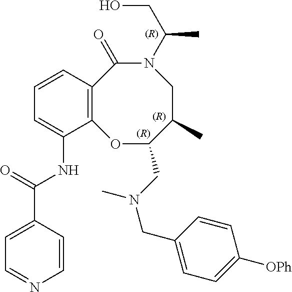

### N-((2R,3R)-5-((R)-1-hydroxypropan-2-yl)-3-methyl-2-((methyl(4-phenoxybenzyl)amino)methyl)-6-oxo-3,4,5,6-tetrahydro-2H-benzo[b][1,5]oxazocin-10-yl)isonicotinamide, 32

Synthesized using the procedure for 9 replacing 3 with the (R,R,R) amine. [M+H]=595.42.

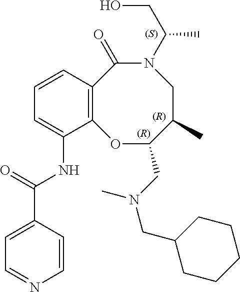

### N-((2R,3R)-2-(((cyclohexylmethyl)(methyl)amino)methyl)-5-((S)-1-hydroxypropan-2-yl)-3-methyl-6-oxo-3,4,5,6-tetrahydro-2H-benzo[b][1,5]oxazocin-10-yl)isonicotinamide, 33

Synthesized using the procedure for 9 replacing 4-phenoxybenzaldehyde with cyclohexyl aldehyde. [M+H]=509.16.

### Biological Assays

A high-throughput screen of small molecules was performed in duplicate in the recombinant Tulahuen strain of T. cruzi stably expressing a beta-galactosidase reporter gene. Trypomastogote-stage parasites were co-cultured with mouse fibroblast NIH/3T3 host cells, (Buckner F S, Verlinde C L, La Flamme A C, Van Voorhis W C. Efficient technique for screening drugs for activity against Trypanosoma cruzi using parasites expressing beta-galactosidase. Antimicrob Agents Chemother. 1996; 40(11): 2592-2597). Signal was normalized to neutral (DMSO) controls, and a 75% inhibition cutoff was used to define a hit. Compounds initially designated as hits were retested in dose in the primary assay to confirm their inhibitory activity. In parallel, compounds were tested in toxicity assays against NIH/3T3 host cells to determine if these compounds were cytotoxic to mammalian cells and thus, false positives. Compounds that reduced the viability of NIH/3T3 cells were excluded as viable hits. A subset of hits were then tested in a similar infection assay with the Tulahuen strain of T. cruzi infecting murine L-6 cells and the intracellular T. cruzi immunofluorescence assay described below.

### A. Methods

1. Materials and Methods

The methods in this section were either performed as described in Bettiol et al., Identification of three classes of heteroaramtic compounds with activity against intracellular Trypanosoma cruzi by chemical library screening. LPoS Negl Trop Dis 2009; 3(2); e384. Epub 2009 Feb. 24 (i.e., immunofluorescence) or modified for high throughput screening (co-culture and host cell toxicity).

Assay Materials:
Dulbecco's modified Eagle's medium (DMEM) with Phenol Red, high glucose, with L-glutamine and sodium pyruvate was obtained from Cellgro (Mediatech Inc, Manassas, Va.; Catalog no. 10-013-CM). PSG or Penicillin-streptomycin-L-glutamine (Catalog no. 10378-016), FBS-heat inactivated fetal bovine serum (FBS, Catalog no.16140-089), and 0.25% Trypsin-EDTA 1× (Catalog no. 25200-072) were purchased from Gibco-Invitrogen. Sterile horse serum, from donor herd (if appearance of epimastigotes) was obtained from Sigma (Catalog no. H1270). Sterile, Ca++/Mg++-free Phosphate Buffer Saline (PBS) 1× was prepared in house. GAL-SCREEN® Buffer B was obtained from Applied Biosystems (Carlsbad, Calif.); Catalog no. T1031). Alexa Fluor 488 goat anti-rabbit IgG secondary antibody was from MOLECULAR PROBES®, Invitrogen (Carlsbad, Calif.). Polyclonal rabbit anti-T cruzi was a gift from Dr. B. Burleigh, Harvard School of Public Health, Boston, Mass.).

Cell Lines:
The following cell lines were used in this study: LLC-MK2 cells (rhesus monkey kidney epithelial cell line) and NIH/3T3 cells (mouse embryonic fibroblastic cell line) were obtained from ATCC. T cruzi expressing β-galactosidase (T cruzi-β-gal: Tulahuen strain, clone C4; refer to Buckner et al., 1996).

### B. Assays

1. T. cruzi Inhibition Assay

### For Cell Propagation:

90% DMEM, Phenol Red, 10% FBS, and 1% PSG were mixed and filtered through a 0.2 microns membrane. The cells were kept at 4° C., then warmed up to 37° C. in a water bath before use.

For T. cruzi Culture and Assays:

98% DMEM, Phenol Red, 2% FBS, and 1% PSG were mixed and filtered through a 0.2 microns membrane. The cells were kept at 4° C., then warmed up to 37° C. in a water bath before use.

### Solutions:

Gal-Screen. Using a Gal-Screen base kit, Buffer B (Catalog no. T2361) was mixed with 1:25 substrate (Catalog no. T2359).

### NIH/3T3 Cell Culture:

NIH/3T3 cells were cultivated in DMEM supplemented with 10% FBS and 1% PSG in T175 in 50 mL total of medium.

### LLC-MK2 Cell Culture:

LLC-MK2 cells were cultivated in DMEM supplemented with 10% FBS and 1% PSG in T175 flasks in 50 mL total of medium. Cells were usually passaged twice a week at 1:4 to 1:8 ratios.

Parasite Culture: T. cruzi β-gal (Tc): T. cruzi β-gal were cultivated in DMEM supplemented with 2% FBS and 1% PSG in T175 flasks with vented caps in 50 mL total of medium.

2. Growth Inhibition Assay for HTS (384-Well Plates)

The medium was warmed up with 2% FBS/DMEM. The parasites were harvested in 50-mL tubes, and spun for 10 minutes at 2200 rpm. Approximately 15 mL of media was aspirated, and the samples were incubated for 3-5 h. The NIH/3T3 cells were trypsinized (refer to cell culture protocol). When the NIH/3T3 cells were detached, the cells were harvested in DMEM, 2% FBS, and 1% PSG, then counted using the Nexcelom cellometer. The cells were diluted to 166,667 cells/mL, then added to a flask and plated 5,000 cells/30 μL per well using a standard cassette multiwall drob Combi. The cells were incubated for 3 h, then T. cruzi cells were counted, diluted to 0.166 million cells/mL, and transferred to a 2-liter flask. Then, 100 mL compounds/DMSO were pinned to each well with NIH/3T3 cells. Next, 30 μL/well of parasites (5000 T. cruzi) were added with a standard cassette multiwall drop Combi on slow speed, and incubated for 4 days (or a minimum of 90 h). Gal-Screen was prepared, 30 μL per well were dispensed in a 384-well plate, incubated for 60 minutes, and the luminescence was read using Envision (Perkin-Elmer) at 0.1 sec/well.

3. Cell Toxicity Assay: NIH/3T3 Cells

For the cell toxicity assay with NIH/3T3 cells, the same materials as for T. cruzi co-culture assay were used. NIH/3T3 cells were cultivated in DMEM supplemented with 10% FBS and 1% PSG in T175 in 50 mL total of medium.

4. Intracellular T cruzi Immunofluorescence Assay

Fifty thousand NIH/3T3 cells were seeded on sterile glass coverslips in 12-well plates and allowed to adhere overnight. Five million T cruzi parasites were added (multiplicity of infection 100:1) and allowed to infect for 2 h in DMEM+2% FBS and PSG. Parasites were rinsed out 3× with PBS, and compounds were added at 10× their IC₅₀ (as determined in AID 2044 and AID 2294). Infected cells were further incubated for 4 days and fixed for 15 min with 4% paraformaldehyde.

Fixed cells on coverslips were rinsed with PBS and permeabilized for 15 min in PBS with 0.1% Triton X-100. After blocking for 20 min in PBS with 10% goat serum, 1% bovine serum albumin (BSA), 100 mM glycine, and 0.05% sodium azide, cells were incubated for 1 h at room temperature with a polyclonal rabbit anti-T cruzi at 1:2000 dilution. After rinsing, an Alexa Fluor 488 goat anti-rabbit IgG secondary antibody was added for 1 h at a 1:800 dilution. DNA was stained with DAPI, and coverslips were mounted with anti-fade mounting media. Images were taken using an inverted Olympus IX70 microscope with a 60× oil objective.

5. Counter Screens

In vitro counter screens were performed to confirm our findings. The results of these screens for compounds of Table 1 are summarized in Table 3.

TABLE 3

| | T. b. rhod. | T. cruzi | | P. falc. K1 | Cytotox. | | | | L. don. | P. falc. | |
|---|---|---|---|---|---|---|---|---|---|---|---|
| Comp. | (IC50, | IC50 | L. don. axen. | IC50 | L6 IC50 | Mol. | T. b. rhod | T. cruzi | Axen (IC50 | K1 | cytotox, |
| Number | ug/mL) | (ug/mL) | IC50 (ug/mL) | (ug/mL) | (ug/mL) | Weight | (IC50 uM) | (IC50 uM) | uM) | (IC50 uM) | (IC50 uM) |
| 16 | 2.62 | 0.225 | >100 | 5.91 | >100 | 533 | 4.92 | 0.422 | >188 | 11.1 | >188 |
| 4 | 6.86 | 0.078 | >100 | 5.79 | >100 | 547 | 12.5 | 0.143 | >183 | 10.6 | >183 |
| 19 | 5.33 | 0.205 | >100 | 4.77 | >100 | 503 | 10.6 | 0.408 | >199 | 9.49 | >199 |
| 1 | 75.4 | 0.622 | >100 | 20.6 | >100 | 503 | 150 | 1.24 | >199 | 41 | >199 |
| 3 | 2.63 | 1.96 | >100 | 3.22 | 44.2 | 595 | 4.42 | 3.3 | >168 | 5.41 | 74.3 |
| 10 | 6.15 | 0.129 | >100 | 2.78 | 43.7 | 595 | 10.3 | 0.217 | >168 | 4.67 | 73.5 |
| 7 | 0.729 | 0.071 | 13.3 | 1.2 | 13.5 | 579 | 1.26 | 0.123 | 23 | 2.07 | 23.3 |
| 13 | 3.49 | 0.499 | 16.3 | 2.95 | 45.5 | 571 | 6.12 | 0.875 | 28.6 | 5.17 | 79.7 |
| 6 | 7.22 | 0.497 | 41.4 | 3.79 | 36.1 | 571 | 12.7 | 0.871 | 72.6 | 6.64 | 63.3 |
| 9 | 35.1 | 0.153 | 84.4 | 4.83 | >100 | 503 | 69.8 | 0.304 | 168 | 9.61 | >199 |
| 8 | 15.4 | 0.278 | >100 | 8.41 | >100 | 533 | 28.9 | 0.522 | >188 | 15.8 | >188 |
| 5 | 20.2 | 0.699 | >100 | 7.39 | >100 | 533 | 37.9 | 1.31 | >188 | 13.9 | >188 |
| 20 | 6.41 | 0.228 | 6.47 | 1.14 | 17.5 | 579 | 11.1 | 0.394 | 11.2 | 1.97 | 30.2 |
| 18 | 11.2 | 0.628 | >100 | 3.21 | 47.5 | 579 | 19.4 | 1.09 | >173 | 5.55 | 82.1 |
| 11 | 0.715 | 0.029 | 21.2 | 1.15 | 10.8 | 595 | 1.2 | 0.0488 | 35.6 | 1.93 | 18.2 |
| 14 | 18.5 | 0.318 | >100 | 1.82 | 52.2 | 595 | 31.1 | 0.535 | >168 | 3.06 | 87.8 |
| 17 | 6.14 | 0.196 | >100 | 5.64 | >100 | 503 | 12.2 | 0.39 | >199 | 11.2 | >199 |
| 2 | 11.1 | 2.26 | >100 | 5.59 | >100 | 571 | 19.4 | 3.95 | >175 | 9.78 | >175 |
| 15 | 5.78 | 0.193 | 13.2 | 1.02 | 14.4 | 595 | 9.72 | 0.325 | 22.2 | 1.72 | 24.2 |
| 12 | 51.5 | 0.41 | >100 | 19.2 | >100 | 503 | 102 | 0.816 | >199 | 38.2 | >199 |

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A compound of Formula II or a pharmaceutically acceptable ester, salt, prodrug or metabolite thereof; 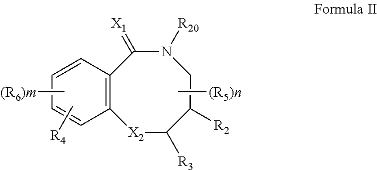
wherein
n is 0, 1, 2, 3, 4 or 5;
m is 0, 1, 2, 3 or 4;
X₁ is O or S;
X₂ is O, S, S(O) or S(O)₂;
R₂₀ is alkyl, alkenyl or alkynyl, preferably C₁-C₄ alkyl;
each R₂, R₃, R₅ and R₆ is independently selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀, —SR₁₀, —NR₁₀R₁₁, —C(O)R₁₀, —C(O)OR₁₀, —C(O)NR₁₀R₁₁, —N(R₁₀)C(O)R₁₁, —CF₃, —CN, —NO₂, —N₃, acyl, alkoxy, substituted alkoxy, alkylamino, substituted alkylamino, dialkylamino, substituted dialkylamino, alkylthio or substituted alkylthio;
wherein each R₁₀ and R₁₁ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₀ and R₁₁ together with the atoms to which they are attached and any intervening atoms may form an additional optionally substituted, 3, 4, 5, 6 or 7 membered ring; and,
R₄ is selected from hydrogen, halogen, alkyl, substituted alkyl, alkenyl, substituted alkenyl, alkynyl, substituted alkynyl, aliphatic, substituted aliphatic, aryl, substituted aryl, —OR₁₀, —SR₁₀, —NR₁₀R₁₁, —C(O)R₁₀, —C(O)OR₁₀, —C(O)NR₁₀R₁₁, —N(R₁₀)C(O)R₁₁, —CF₃, —NO₂, —N₃.

2. A compound of Formula III or a pharmaceutically acceptable ester, salt, prodrug or metabolite thereof; 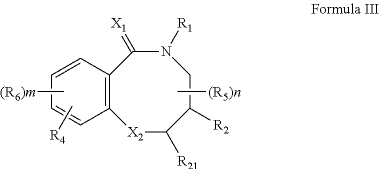
wherein R₁ is hydrogen, alkyl, substituted alkyl, alkenyl, substituted alkenyl, alkynyl, substituted alkynyl, alkoxy, substituted alkoxy, alkylhydroxy, substituted alkylhydroxy, alkylamino, substituted alkylamino, alkylthio or substituted alkylthio;
R₂₁ is -G₂₀-X₂₀;
wherein G₂₀ is absent, C₁-C₁₂-alkyl, substituted C₁-C₁₂-alkyl, C₂-C₁₂-alkenyl, substituted C₂-C₁₂-alkenyl, C₂-C₁₂-alkynyl or substituted C₂-C₁₂-alkynyl; and,
X₂₀ is —NR₃₀R₃₁, —OR₃₀, —SR₃₀, —C(O)R₃₀, —C(O)OR₃₀, —C(O)NR₃OR₃₁, —N(R₃₀)C(O)R₃₁, —S(O)R₃₀ or S(O)₂R₃₁;
wherein R₃₀ is selected from absent, hydrogen, alkyl, substituted alkyl, alkenyl, substituted alkenyl, alkynyl, substituted alkynyl, aliphatic, substituted aliphatic, aryl and substituted aryl; and,
R₃₁ is aryl substituted with 1, 2, 3, 4 or 5 halogens, or heteroaryl substituted with 1, 2, 3, 4 or 5 halogens.

3. A compound of Formula V or a pharmaceutically acceptable ester, salt, prodrug or metabolite thereof; 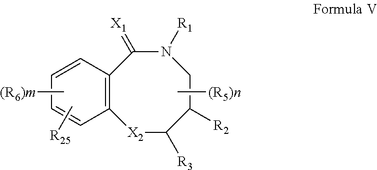
wherein R₂₅ is aryl, substituted aryl, heteroaryl, substituted heteroaryl, heterocyclic or substituted heterocyclic.

4. A compound of Formula IA or a pharmaceutically acceptable ester, salt, prodrug or metabolite thereof;

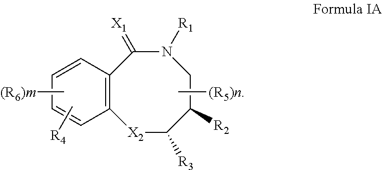

5. A compound according to claim 1, wherein X₁ is O.

6. A compound according to claim 1, wherein X₂ is O.

7. A compound according to claim 1, wherein R₂ is selected from hydrogen, halogen, C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₅-C₁₂ aryl, substituted C₅-C₁₂ aryl C₃-C₁₂ cycloalkyl and substituted C₃-C₁₂-cycloalkyl.

8. A compound according to claim 1, wherein R₄ is —X₄-G₂;
wherein X₄ is absent, C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, —NR₁₄—, —O—, —S—, —C(O)—, —C(O)O—, —C(O)NR₁₄—, —N(R₁₄)C(O)—, —S(O)— or S(O)₂—; wherein R₁₄ is selected from hydrogen, alkyl, substituted alkyl, alkenyl, substituted alkenyl, alkynyl, substituted alkynyl, aliphatic, substituted aliphatic, aryl and substituted aryl; and,
G₂ is aryl or substituted aryl.

9. A compound according to claim 1, wherein R₄ is —X₄-G₂ wherein —X₄— is —NHC(O)—, —C(O)N(CH₃)— —C(O)—, —C(O)O— or —NH—.

10. A compound according to claim 1, wherein G₂ is selected from Table B:
TABLE B

| | |
|---|---|
| 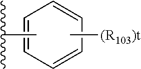 | 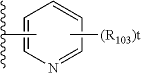 |
| 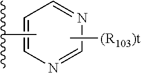 | 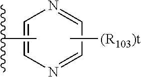 |
| 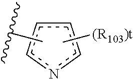 | 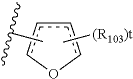 |
| 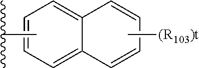 | 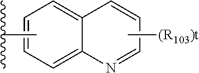 |
| 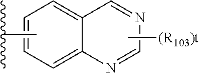 | 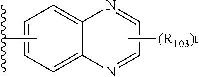 |

t is 0, 1, 2, 3, 4 or 5;
R₁₀₃ is selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀₁, —SR₁₀₄, —NR₁₀₄R₁₀₅, —C(O)R₁₀₄, —C(O)OR₁₀₄, —C(O)NR₁₀₄R₁₀₅, —N(R₁₀₄)C(O)R₁₀₅, —CF₃, —CN, —NO₂, —N₃, acyl, alkoxy, substituted alkoxy, alkylamino, substituted alkylamino, dialkylamino, substituted dialkylamino, alkylthio or substituted alkylthio;
wherein each R₁₀₄ and R₁₀₅ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two R₁₀₄ and R₁₀₅ groups together with the atoms to which they are attached and any intervening atoms may form an additional optionally substituted, 3, 4, 5, 6 or 7 membered ring.

11. A compound according to claim 3, wherein R₂₅ is selected from:

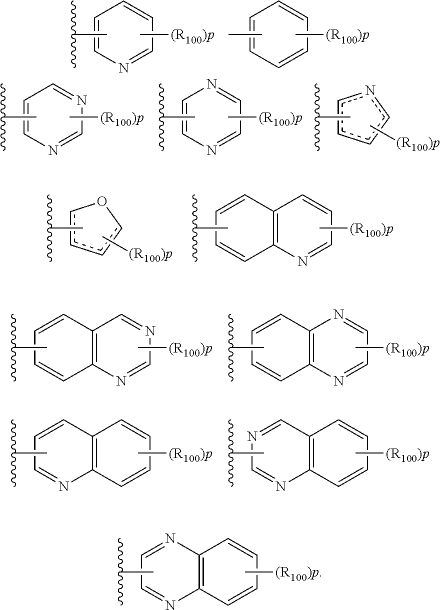

12. A method of treating a parasitic infection or a disease or disorder caused by a parasitic infection comprising the step of administering a compound of Formula I to a subject in need thereof: 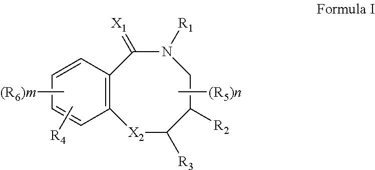
wherein
n is 0, 1, 2, 3, 4 or 5;
m is 0, 1, 2, 3 or 4;
X₁ is O or S;
X₂ is O, S, S(O) or S(O)₂;
R₁ is hydrogen, alkyl, substituted alkyl, alkenyl, substituted alkenyl, alkynyl, substituted alkynyl, alkoxy, substituted alkoxy, alkylhydroxy, substituted alkylhydroxy, alkylamino, substituted alkylamino, alkylthio or substituted alkylthio;
each R₂, R₃, R₅ and R₆ is independently selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀, —SR₁₀, —NR₁₀R₁₁, —C(O)R₁₀, —C(O)OR₁₀, —C(O)NR₁₀R₁₁, —N(R₁₀)C(O)R₁₁, —CF₃, —CN, —NO₂, —N₃, acyl, alkoxy, substituted alkoxy, alkylamino, substituted alkylamino, dialkylamino, substituted dialkylamino, alkylthio or substituted alkylthio;
wherein each R₁₀ and R₁₁ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₀ and R₁₁ together with the atoms to which they are attached and any intervening atoms may form an additional optionally substituted, 3, 4, 5, 6 or 7 membered ring; and,
R₄ is selected from hydrogen, halogen, alkyl, substituted alkyl, alkenyl, substituted alkenyl, alkynyl, substituted alkynyl, aliphatic, substituted aliphatic, aryl, substituted aryl, —OR₁₀, —SR₁₀, —NR₁₀R₁₁, —C(O)R₁₀, —C(O)OR₁₀, —C(O)NR₁₀R₁₁, —N(R₁₀)C(O)R₁₁, —CF₃, —CN, —NO₂, —N₃.

13. The method according to claim 12, wherein X₁ is O.

14. The method according to claim 12, wherein X₂ is O.

15. The method according to claim 12, wherein R₁ is selected from C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₁-C₁₂-alkylhydroxy, substituted C₁-C₁₂-alkylhydroxy, C₂-C₁₂-alkenylhydroxy, substituted C₂-C₁₂-alkenylhydroxy, C₂-C₁₂-alkynylhydroxy, substituted C₂-C₁₂-alkynylhydroxy, C₁-C₁₂-alkylthio, substituted C₁-C₁₂-alkylthio, C₂-C₁₂-alkenylthio, substituted C₂-C₁₂-alkenylthio, C₂-C₁₂-alkynylthio, substituted C₂-C₁₂-alkynylthio, C₃-C₁₂-cycloalkyl and substituted C₃-C₁₂-cycloalkyl.

16. The method according to claim 12, wherein R₂ is selected from hydrogen, halogen, C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₅-C₁₂ aryl, substituted C₅-C₁₂ aryl C₃-C₁₂-cycloalkyl and substituted C₃-C₁₂-cycloalkyl.

17. The method according to claim 12, wherein R₃ is -G₁-X₃;
wherein G₁ is absent, C₁-C₁₂-alkyl, substituted C₁-C₁₂-alkyl, C₂-C₁₂-alkenyl, substituted C₂-C₁₂-alkenyl, C₂-C₁₂-alkynyl or substituted C₂-C₁₂-alkynyl; and,
X₃ is —NR₁₂R₁₃, —OR₁₂, —SR₁₂, —C(O)R₁₂, —C(O)OR₁₂, —C(O)NR₁₂R₁₃, —N(R₁₂)C(O)R₁₃, —S(O)R₁₂ or S(O)₂R₁₂;
wherein each R₁₂ and R₁₃ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₂ and R₁₃ together with the atoms to which they are attached and any intervening atoms may form an additional optionally substituted, 3, 4, 5, 6 or 7 membered ring.

18. The method according to claim 12, wherein R₁₂ is selected from Table A: 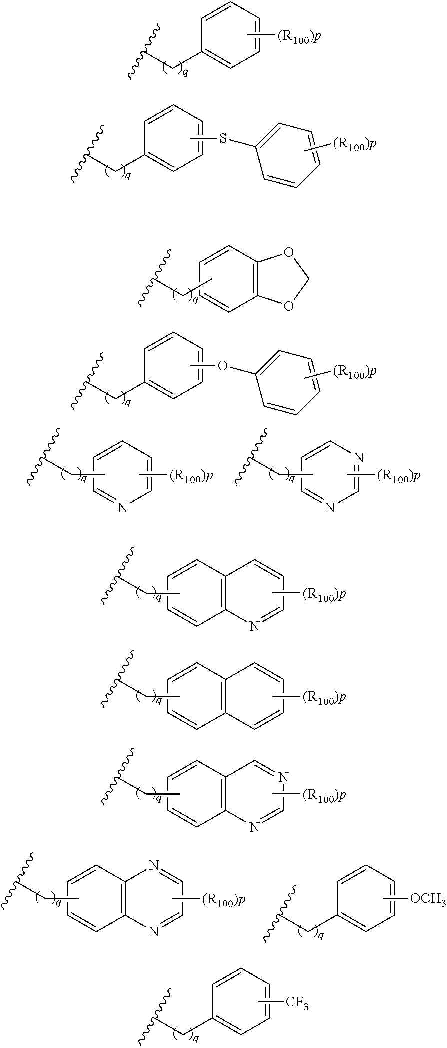
wherein q is 0, 1, 2, 3, 4, 5, or 6;
p is 0, 1, 2, 3, 4 or 5;
R₁₀₀ is selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀₁, —SR₁₀₁, —NR₁₀₁R₁₀₂, —C(O)R₁₀₁, —C(O)OR₁₀₁, —C(O)NR₁₀₁R₁₀₂, —N(R₁₀₁)C(O)R₁₀₂, —CF₃, —CN, —NO₂, —N₃, acyl, alkoxy, substituted alkoxy, alkylamino, substituted alkylamino, dialkylamino, substituted dialkylamino, alkylthio or substituted alkylthio;
wherein each R₁₀₁ and R₁₀₂ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two of R₁₀₁ and R₁₀₂ together with the atoms to which they are attached and any intervening atoms may form an additional optionally substituted, 3, 4, 5, 6 or 7 membered ring.

19. The method according to claim 12, wherein G₂ is selected from Table B: 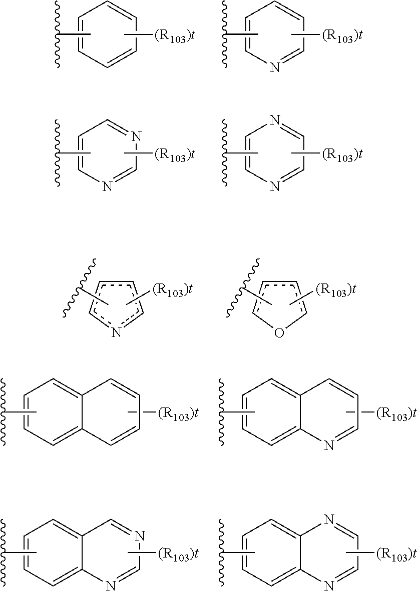
wherein t is 0, 1, 2, 3, 4 or 5;
R₁₀₃ is selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀₁, —SR₁₀₄, —NR₁₀₄R₁₀₅, —C(O)R₁₀₄, —C(O)OR₁₀₄, —C(O)NR₁₀₄R₁₀₅, —N(R₁₀₄)C(O)R₁₀₅, —CF₃, —CN, —NO₂, —N₃, acyl, alkoxy, substituted alkoxy, alkylamino, substituted alkylamino, dialkylamino, substituted dialkylamino, alkylthio or substituted alkylthio;
wherein each R₁₀₄ and R₁₀₅ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two R₁₀₄ and R₁₀₅ groups together with the atoms to which they are attached and any intervening atoms may form an additional optionally substituted, 3, 4, 5, 6 or 7 membered ring.

20. The method according to claim 12, wherein R₁ is selected from Table C: 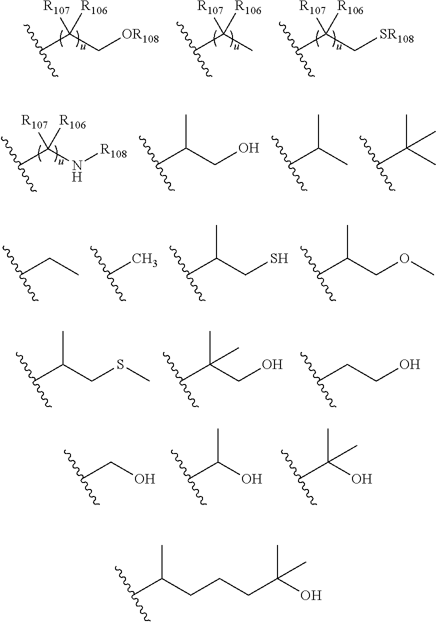
wherein u is 0, 1, 2, 3, 4, 5, 6, 7, 8, 9 or 10;
each R₁₀₆, R₁₀₇ and R₁₀₈ is independently selected from absent, hydrogen, halogen, alkyl, substituted alkyl, alkenyl, substituted alkenyl, alkynyl, substituted alkynyl, aliphatic, substituted aliphatic, aryl, substituted aryl, —OR₁₀₉, —SR₁₀₉, —NR₁₀₉R₁₁₀, —C(O)R₁₀₉, —C(O)OR₁₀₉, —C(O)NR₁₀₉R₁₁₀, —N(R₁₀₉)C(O)R₁₁₀, —CF₃, —CN, —NO₂, —N₃, acyl, alkoxy, substituted alkoxy, alkylamino, substituted alkylamino, dialkylamino, substituted dialkylamino, alkylthio or substituted alkylthio;
wherein each R₁₀₉ and R₁₁₀ is independently selected from absent, hydrogen, alkyl, substituted alkyl, alkenyl, substituted alkenyl, alkynyl, substituted alkynyl, aliphatic, substituted aliphatic, aryl and substituted aryl; alternatively two R₁₀₉ and R₁₁₀ groups together with the atoms to which they are attached and any intervening atoms may form an additional optionally substituted, 3, 4, 5, 6 or 7 membered ring.

21. The method according to claim 12, wherein said compound is selected from:

| Com- | |
|---|---|
| pound | |
| No. | |
| 1. | 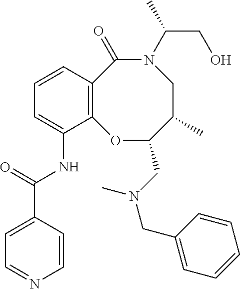 [SMILES: C[C@H](CO)N1C[C@H](C)[C@H](CN(C)Cc2ccccc2)Oc2c(NC(=O)c3ccncc3)cccc2C1=O] |
| 2. | 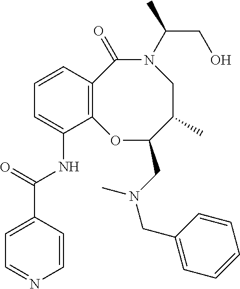 [SMILES: C[C@H]1CN([C@@H](C)CO)C(=O)c2cccc(NC(=O)c3ccncc3)c2O[C@@H]1CN(C)Cc1ccccc1] |
| 3. | 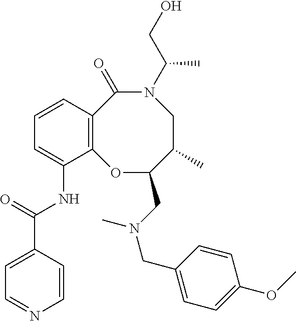 [SMILES: COc1ccc(CN(C)C[C@H]2Oc3c(NC(=O)c4ccncc4)cccc3C(=O)N([C@@H](C)CO)C[C@@H]2C)cc1] |
| 4. | 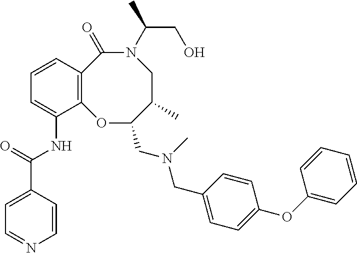 [SMILES: C[C@H]1CN([C@@H](C)CO)C(=O)c2cccc(NC(=O)c3ccncc3)c2O[C@H]1CN(C)Cc1ccc(Oc2ccccc2)cc1] |
| 5. | 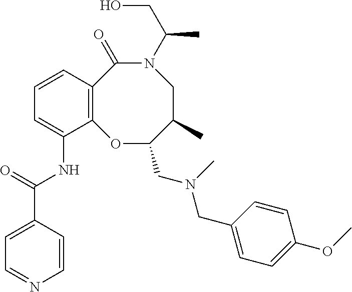 [SMILES: COc1ccc(CN(C)C[C@@H]2Oc3c(NC(=O)c4ccncc4)cccc3C(=O)N([C@H](C)CO)C[C@H]2C)cc1] |
| 6. | 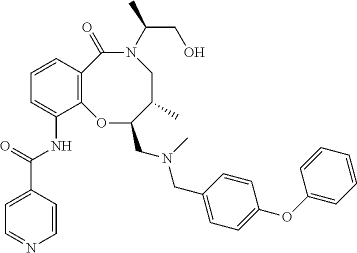 [SMILES: C[C@H]1CN([C@@H](C)CO)C(=O)c2cccc(NC(=O)c3ccncc3)c2O[C@@H]1CN(C)Cc1ccc(Oc2ccccc2)cc1] |
| 7. | 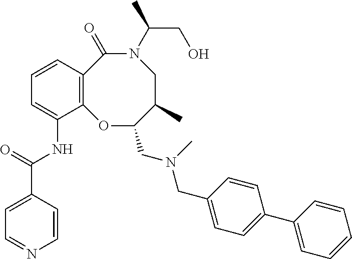 [SMILES: C[C@@H]1CN([C@@H](C)CO)C(=O)c2cccc(NC(=O)c3ccncc3)c2O[C@H]1CN(C)Cc1ccc(-c2ccccc2)cc1] |
| 8. | 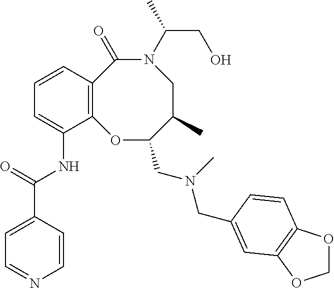 [SMILES: C[C@@H]1CN([C@H](C)CO)C(=O)c2cccc(NC(=O)c3ccncc3)c2O[C@H]1CN(C)Cc1ccc2c(c1)OCO2] |
| 9. | 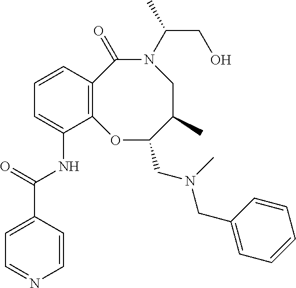 [SMILES: C[C@@H]1CN([C@H](C)CO)C(=O)c2cccc(NC(=O)c3ccncc3)c2O[C@H]1CN(C)Cc1ccccc1] |
| 10. | 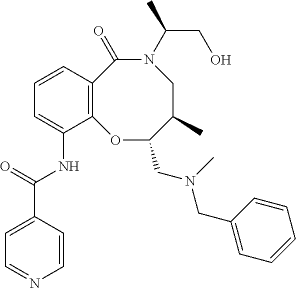 [SMILES: C[C@@H]1CN([C@@H](C)CO)C(=O)c2cccc(NC(=O)c3ccncc3)c2O[C@H]1CN(C)Cc1ccccc1] |
| 11. | 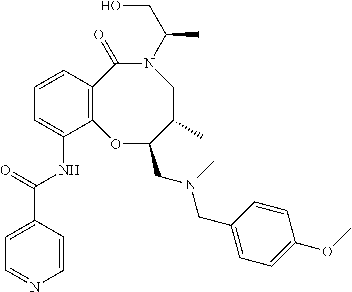 [SMILES: COc1ccc(CN(C)C[C@H]2Oc3c(NC(=O)c4ccncc4)cccc3C(=O)N([C@H](C)CO)C[C@@H]2C)cc1] |
| 12. | 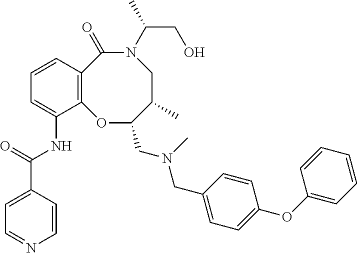 [SMILES: C[C@H](CO)N1C[C@H](C)[C@H](CN(C)Cc2ccc(Oc3ccccc3)cc2)Oc2c(NC(=O)c3ccncc3)cccc2C1=O] |
| 13. | 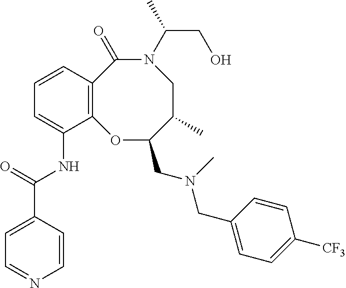 [SMILES: C[C@H](CO)N1C[C@H](C)[C@@H](CN(C)Cc2ccc(C(F)(F)F)cc2)Oc2c(NC(=O)c3ccncc3)cccc2C1=O] |
| 14. | 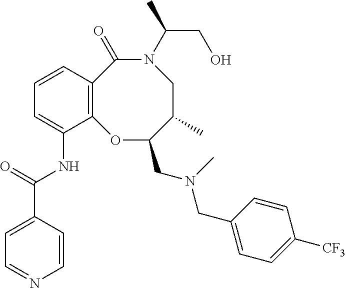 [SMILES: C[C@H]1CN([C@@H](C)CO)C(=O)c2cccc(NC(=O)c3ccncc3)c2O[C@@H]1CN(C)Cc1ccc(C(F)(F)F)cc1] |
| 15. | 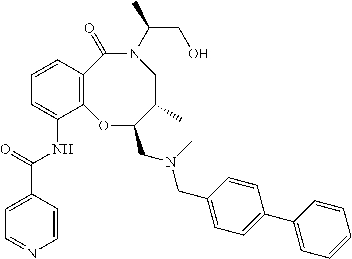 [SMILES: C[C@H]1CN([C@@H](C)CO)C(=O)c2cccc(NC(=O)c3ccncc3)c2O[C@@H]1CN(C)Cc1ccc(-c2ccccc2)cc1] |
| 16. | 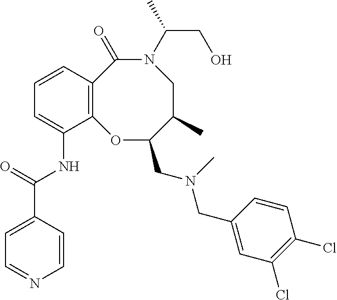 [SMILES: C[C@@H]1CN([C@H](C)CO)C(=O)c2cccc(NC(=O)c3ccncc3)c2O[C@@H]1CN(C)Cc1ccc(Cl)c(Cl)c1] |
| 17. | 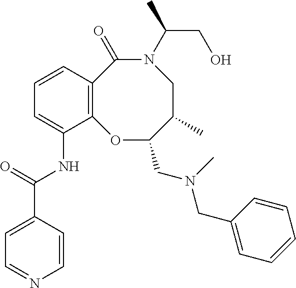 [SMILES: C[C@H]1CN([C@@H](C)CO)C(=O)c2cccc(NC(=O)c3ccncc3)c2O[C@H]1CN(C)Cc1ccccc1] |
| 18. | 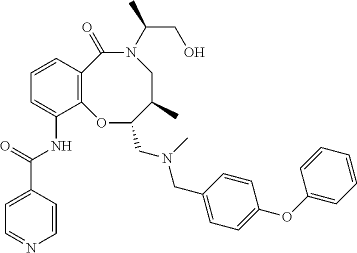 [SMILES: C[C@@H]1CN([C@@H](C)CO)C(=O)c2cccc(NC(=O)c3ccncc3)c2O[C@H]1CN(C)Cc1ccc(Oc2ccccc2)cc1] |
| 19. | 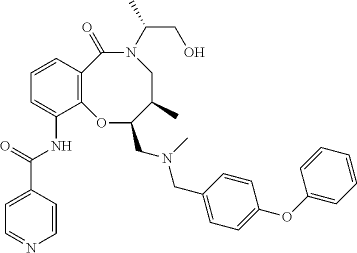 [SMILES: C[C@@H]1CN([C@H](C)CO)C(=O)c2cccc(NC(=O)c3ccncc3)c2O[C@@H]1CN(C)Cc1ccc(Oc2ccccc2)cc1] |
| 20. | 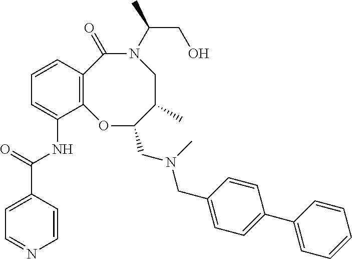 [SMILES: C[C@H]1CN([C@@H](C)CO)C(=O)c2cccc(NC(=O)c3ccncc3)c2O[C@H]1CN(C)Cc1ccc(-c2ccccc2)cc1] |
| 21. | 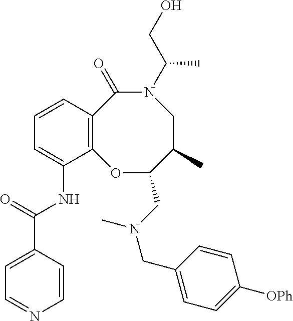 [SMILES: C[C@@H]1CN([C@@H](C)CO)C(=O)c2cccc(NC(=O)c3ccncc3)c2O[C@H]1CN(C)Cc1ccc(Oc2ccccc2)cc1] |
| 22. | 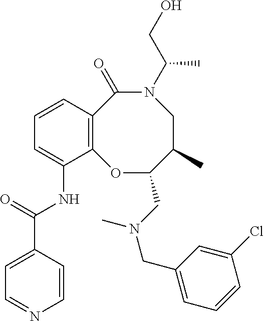 [SMILES: C[C@@H]1CN([C@@H](C)CO)C(=O)c2cccc(NC(=O)c3ccncc3)c2O[C@H]1CN(C)Cc1cccc(Cl)c1] |
| 23. | 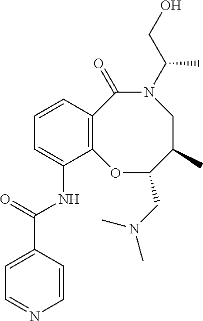 [SMILES: C[C@@H]1CN([C@@H](C)CO)C(=O)c2cccc(NC(=O)c3ccncc3)c2O[C@H]1CN(C)C] |
| 24. | 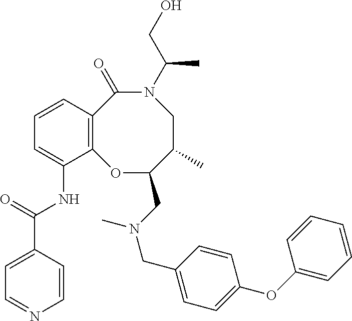 [SMILES: C[C@H](CO)N1C[C@H](C)[C@@H](CN(C)Cc2ccc(Oc3ccccc3)cc2)Oc2c(NC(=O)c3ccncc3)cccc2C1=O] |
| 25. | 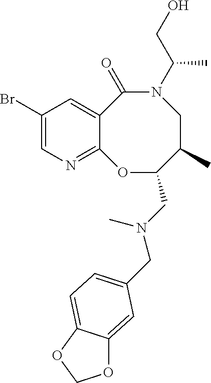 [SMILES: C[C@@H]1CN([C@@H](C)CO)C(=O)c2cc(Br)cnc2O[C@H]1CN(C)Cc1ccc2c(c1)OCO2] |
| 26. | 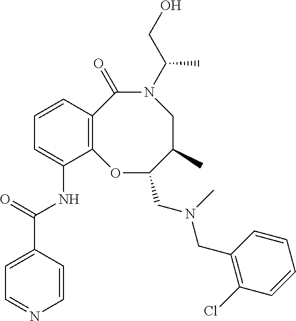 [SMILES: C[C@@H]1CN([C@@H](C)CO)C(=O)c2cccc(NC(=O)c3ccncc3)c2O[C@H]1CN(C)Cc1ccccc1Cl] |
| 27. | 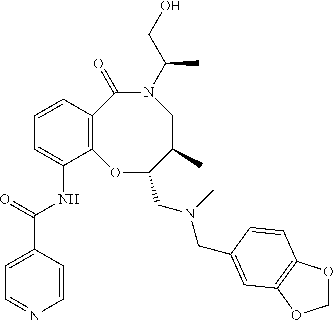 [SMILES: C[C@@H]1CN([C@H](C)CO)C(=O)c2cccc(NC(=O)c3ccncc3)c2O[C@H]1CN(C)Cc1ccc2c(c1)OCO2] |
| 28. | 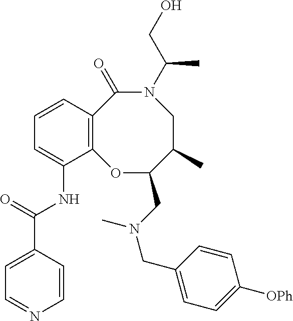 [SMILES: C[C@@H]1CN([C@H](C)CO)C(=O)c2cccc(NC(=O)c3ccncc3)c2O[C@@H]1CN(C)Cc1ccc(Oc2ccccc2)cc1] |
| 29. | 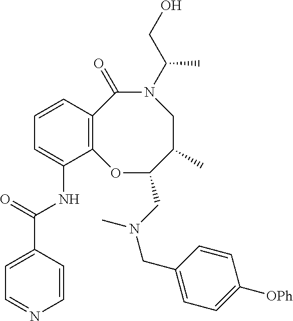 [SMILES: C[C@H]1CN([C@@H](C)CO)C(=O)c2cccc(NC(=O)c3ccncc3)c2O[C@H]1CN(C)Cc1ccc(Oc2ccccc2)cc1] |
| 30. | 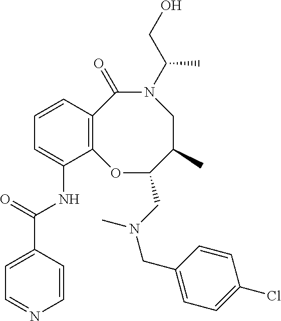 [SMILES: C[C@@H]1CN([C@@H](C)CO)C(=O)c2cccc(NC(=O)c3ccncc3)c2O[C@H]1CN(C)Cc1ccc(Cl)cc1] |
| 31. | 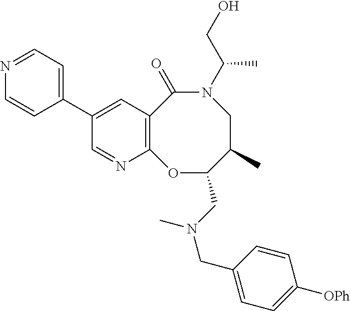 [SMILES: C[C@@H]1CN([C@@H](C)CO)C(=O)c2cc(-c3ccncc3)cnc2O[C@H]1CN(C)Cc1ccc(Oc2ccccc2)cc1] |
| 32. | 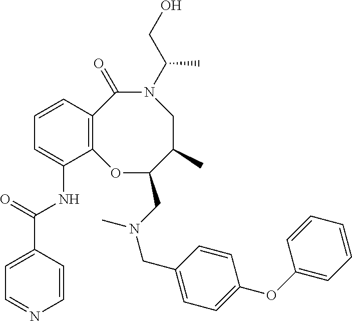 [SMILES: C[C@@H]1CN([C@@H](C)CO)C(=O)c2cccc(NC(=O)c3ccncc3)c2O[C@@H]1CN(C)Cc1ccc(Oc2ccccc2)cc1] |
| 33. | 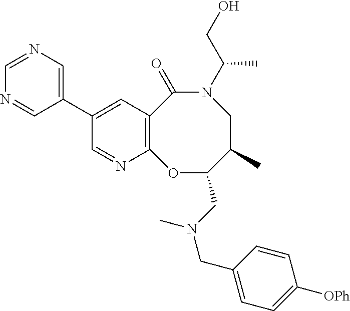 [SMILES: C[C@@H]1CN([C@@H](C)CO)C(=O)c2cc(-c3cncnc3)cnc2O[C@H]1CN(C)Cc1ccc(Oc2ccccc2)cc1] |
| 34. | 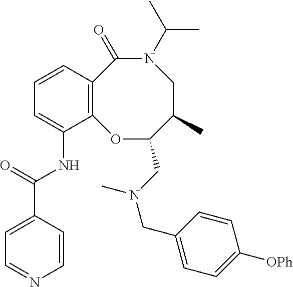 [SMILES: CC(C)N1C[C@@H](C)[C@H](CN(C)Cc2ccc(Oc3ccccc3)cc2)Oc2c(NC(=O)c3ccncc3)cccc2C1=O] |
| 35. | 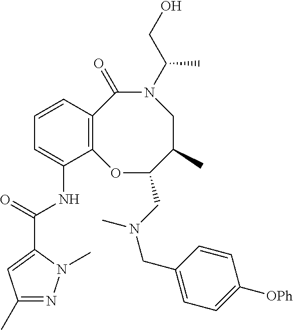 [SMILES: Cc1cc(C(=O)Nc2cccc3c2O[C@@H](CN(C)Cc2ccc(Oc4ccccc4)cc2)[C@H](C)CN([C@@H](C)CO)C3=O)n(C)n1] |
| 36. | 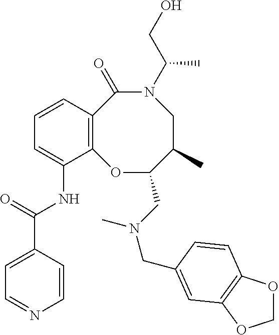 [SMILES: C[C@@H]1CN([C@@H](C)CO)C(=O)c2cccc(NC(=O)c3ccncc3)c2O[C@H]1CN(C)Cc1ccc2c(c1)OCO2] |
| 37. | 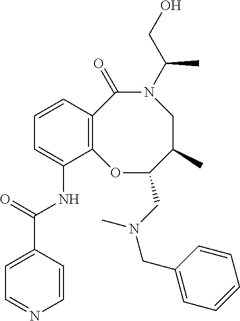 [SMILES: C[C@@H]1CN([C@H](C)CO)C(=O)c2cccc(NC(=O)c3ccncc3)c2O[C@H]1CN(C)Cc1ccccc1] |
| 38. | 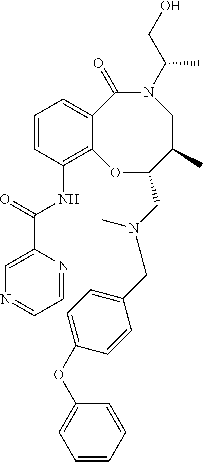 [SMILES: C[C@@H]1CN([C@@H](C)CO)C(=O)c2cccc(NC(=O)c3cnccn3)c2O[C@H]1CN(C)Cc1ccc(Oc2ccccc2)cc1] |
| 39. | 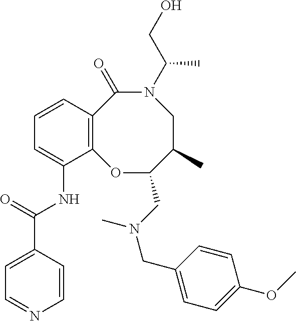 [SMILES: COc1ccc(CN(C)C[C@@H]2Oc3c(NC(=O)c4ccncc4)cccc3C(=O)N([C@@H](C)CO)C[C@H]2C)cc1] |
| 40. | 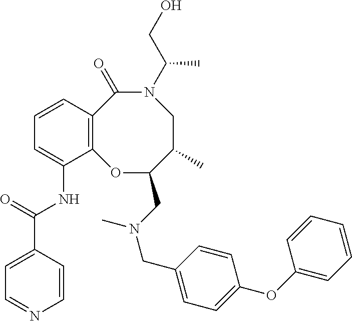 [SMILES: C[C@H]1CN([C@@H](C)CO)C(=O)c2cccc(NC(=O)c3ccncc3)c2O[C@@H]1CN(C)Cc1ccc(Oc2ccccc2)cc1] |
| 41. | 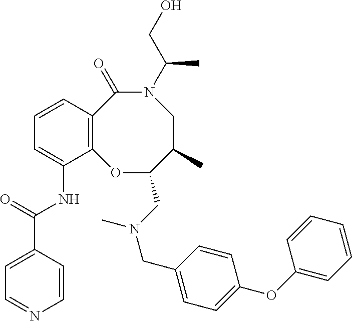 [SMILES: C[C@@H]1CN([C@H](C)CO)C(=O)c2cccc(NC(=O)c3ccncc3)c2O[C@H]1CN(C)Cc1ccc(Oc2ccccc2)cc1] |
| 42. | 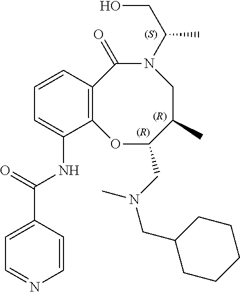 |
| 43. | 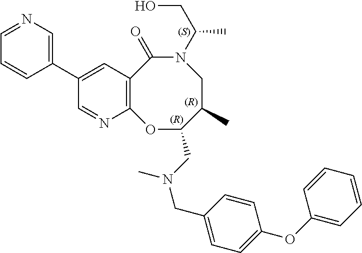 |

22. The method according to claim 12, wherein said parasitic infection, disease or disorder is caused by Trypanosomatids infection or Plasmodium falciparum infection.

23. The method according to claim 22, wherein said disease is South American trypanosomiasis (Chagas disease) or Malaria.

24. A method of treating a disease or disorder caused by Trypanosoma cruzi comprising the step of administering a compound of Formula I, IA, II, III, IV or V to a patient in need thereof.
